(19) United States
(12) Patent Application Publication
(10) Pub. No.: US 2018/0369238 A1
(43) Pub. Date: Dec. 27, 2018

(54) DOPAMINE AGONISTS IN TREATING ALCOHOL USE DISORDERS ASSOCIATED WITH DOPAMINE RECEPTOR ACTIVITY

(71) Applicant: MUSC Foundation for Research Development, Charleston, SC (US)

(72) Inventors: Raymond F. Anton, Charleston, SC (US); Joseph P. Schacht, Charleston, SC (US)

(73) Assignee: MUSC Foundation for Research Development, Charleston, SC (US)

(21) Appl. No.: 16/017,781

(22) Filed: Jun. 25, 2018

Related U.S. Application Data
(60) Provisional application No. 62/524,407, filed on Jun. 23, 2017.
(60) Provisional application No. 62/591,448, filed on Nov. 28, 2017.

(51) Int. Cl. A61K 31/496; A61P 25/32

(52) U.S. Cl. CPC A61K 31/496; A61P 25/32; C12Q 1/6883; C12Q 2600/106; C12Q 2600/156

(57) ABSTRACT

Disclosed are methods for treating disorders associated with dopamine receptor activity. In some embodiments, the disclosed methods include assaying the nucleic acid from a subject for the genotype of the variable number tandem repeats (VNTR) polymorphism in the dopamine transporter DAT1/SLC6A3 gene, wherein when one or two alleles for 9 tandem repeats is detected a dopamine partial agonist is administered to the subject; and wherein when two alleles for 10 tandem repeats is detected a dopamine modulator is not administered to the subject. Also provided are methods for treating disorders associated with dopamine receptor activity that include genotyping a subject with respect to a COMT polymorphism, a DRD2 polymorphism, a 48-base-pair VNTR polymorphism in DRD4 exon 3, and/or a ANKK1 TaqA1 polymorphism, and methods for detecting susceptibility to dopamine modulator therapy for conditions associated with dopamine receptor activity.

### CROSS REFERENCE TO RELATED APPLICATIONS

This application claims the benefit of U.S. Provisional Application Ser. Nos. 62/524,407, filed Jun. 23, 2017, and 62/591,448, filed Nov. 28, 2017. The disclosure of each of these applications is incorporated herein by reference in its entirety.

### GRANT STATEMENT

This invention was made with government support under K99/R00 AA021419, K05 AA017435, and P50 AA010761 awarded by the National Institute on Alcohol Abuse and Alcoholism. The government has certain rights in the invention.

### TECHNICAL FIELD

The presently disclosed subject matter relates in some embodiments to methods for treating disorders associated with dopamine receptor activity. In particular, it relates to genotyping subjects with respect to polymorphisms and tandem repeats and on the basis of the genotyping, either administering or not administering dopamine modulators. The presently disclosed subject matter also relates in some embodiments to methods for detecting susceptibility to dopamine modulator therapy for disorders associated with dopamine receptor activity.

### BACKGROUND

Dopamine (DA) signaling regulates many biological activities, and is associated with several psychiatric, mental, and/or neurological disorders including but not limited to Alcohol Use Disorder (AUD). Alcohol cues and intravenous alcohol self-administration both increase DA release in the human ventral striatum (VS). Relative to controls, individuals with AUD display enhanced alcohol-induced, but blunted amphetamine-induced, VS DA release, and, unlike controls, demonstrate no association between striatal DA release and prefrontal glucose metabolism, suggesting impaired cortical modulation of VS DA signaling. To remediate this impairment, several dopaminergic medications have been explored as AUD treatments, including the atypical antipsychotic aripiprazole (APZ), a high-affinity D₂ and 5-HT_{2B} partial agonist. Positron emission tomography suggests that APZ can stabilize dysregulated DA neurotransmission by increasing striatal DA synthesis among individuals with low basal synthesis capacity, and decreasing it among individuals with high basal capacity.

APZ has been reported to reduce the euphoric effects of alcohol drinking in either the natural environment or in a bar-lab setting as well as alcohol cue-elicited VS activation. A large multisite AUD clinical trial found that APZ did not significantly change the primary drinking outcome (measured as percent days abstinent), but did significantly improve other outcomes, including drinks per drinking day and an alcohol consumption biomarker.

What is needed are methods for treating disorders associated with dopamine receptor activity with APZ and/or other DA modulators that remove these inconsistencies as well as methods for detecting susceptibility of subjects who have disorders associated with dopamine receptor activity to treatment with APZ and/or other DA modulators.

### SUMMARY

This summary lists several embodiments of the presently disclosed subject matter, and in many cases, lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this Summary does not list or suggest all possible combinations of such features.

Disclosed are methods and compositions related to the use of dopamine modulators in the treatment of psychiatric, mental, and/or neurological disorders.

In some embodiments, the presently disclosed subject matter provides methods for treating a psychiatric, mental, and/or neurological disorder (such as, for example, alcohol use disorder (AUD)). In some embodiments, the presently disclosed methods comprise assaying the nucleic acid from a subject for the genotype of the variable number tandem repeats (VNTR) polymorphism in the dopamine transporter gene DAT1/SLC6A3, wherein when one or two alleles for nine (9) tandem repeats is detected, a dopamine modulator is administered to the subject; and wherein when two alleles for ten (10) tandem repeats is detected, a dopamine modulator is not administered to the subject. In some embodiments, the genotype of the DAT1/SLC6A3 VNTR of the subject is assayed prior to administering a dopamine modulator. In some embodiments, the genotype of the DAT1/SLC6A3 VNTR of the subject is assayed after dopamine modulator therapy has commenced, and wherein when two alleles for ten (10) tandem repeats is detected a dopamine modulator therapy is discontinued. In some embodiments, the dopamine modulator is a dopamine agonist (such as but not limited to aripiprazole, brexipiprizole, and/or cariprazine). In some embodiments, the genotype of the VNTR polymorphism is detected by a nucleic acid amplification process, which in some embodiments is followed by sequencing, gel electrophoresis, direct sequencing, or any combination thereof.

In some embodiments, the presently disclosed methods further comprise assaying for one or more polymorphisms in the genes encoding the DA-catabolizing enzyme catechol-O-methyltransferase (COMT), the D₂ receptor (DRD2), the D₄ receptor (DRD4), or any combination thereof.

The presently disclosed subject matter also provides in some embodiments methods for detecting susceptibility to dopamine modulator therapy for a psychiatric, mental, and/or neurological disorder (such as, for example, alcohol use disorder (AUD)). In some embodiments, the presently disclosed methods comprise obtaining a biological sample from a subject and assaying nucleic acid from the biological sample from the subject for the genotype of the variable number tandem repeats (VNTR) polymorphism in the dopamine transporter gene DAT1/SLC6A3, wherein detection of one or two alleles for nine (9) tandem repeats indicates that the subject is susceptible to dopamine modulator therapy. In some embodiments, the dopamine modulator is a dopamine agonist (such as but not limited to aripiprazole, brexipiprizole, and/or cariprazine). In some embodiments, the genotype of the VNTR polymorphism is detected by a nucleic acid amplification process followed by sequencing, gel electrophoresis, direct sequencing, or any combination thereof.

More particularly, in some embodiments the presently disclosed subject matter provides methods for treating subjects with disorders associated with dopamine receptor activity, which in some embodiments can comprise performing or having performed one or more genotyping assays on a nucleic acid sample isolated from the subject to determine the subject's genotype with respect to a variable number tandem repeats (VNTR) polymorphism in a dopamine transporter DAT1/SLC6A3 gene, an rs4680 polymorphism in a DA-catabolizing enzyme catechol-O-methyltransferase (COMT) gene, an rs1076560 polymorphism in a D2 receptor (DRD2) gene, a 48-base-pair VNTR polymorphism in a D4 receptor (DRD4) gene, and/or an rs1800497 polymorphism in an ankyrin repeat and kinase domain containing 1 (ANKK1) gene; and administering a dopamine partial agonist to the subject if the one or more genotyping assays indicates that subject's genotype includes at least one allele for 9 tandem repeats of the DAT1/SLC6A3 VNTR; or four or more of a 9 tandem repeat allele of the DAT1/SLC6A3 VNTR; a COMT A allele of the rs4680 polymorphism; a 48-base-pair VNTR in DRD4 exon 3 allele; and a DRD2 T allele of the rs1076560 polymorphism, an ANKK1 TaqA1 A allele of the rs1800497 polymorphism, or both. In some embodiments, the disorder associated with dopamine receptor activity is an alcohol use disorder (AUD). In some embodiments, the one or more genotyping assays are performed prior to administering the dopamine partial agonist. In some embodiments, the one or more genotyping assays are performed after initiating a dopamine partial agonist therapy, and further wherein if the subject is homozygous for a DAT1/SLC6A3 VNTR 10 tandem repeat allele, the dopamine partial agonist therapy is discontinued. In some embodiments, the dopamine partial agonist is selected from the group consisting of aripiprazole, brexipiprizole, and cariprazine. In some embodiments, at least one of the one or more genotyping assays comprises a nucleic acid amplification process followed by sequencing or gel electrophoresis of a resulting nucleic acid amplification product. In some embodiments, the one or more genotyping assays determine the subject's genotype with respect to the VNTR polymorphism in the dopamine transporter DAT1/SLC6A3 gene, the rs1076560 polymorphism in the DRD2 gene, and the 48-base-pair VNTR polymorphism in the DRD4 gene. In some embodiments, the one or more genotyping assays further comprise a genotyping assay that determines the subject's genome with respect to the rs4680 polymorphism in the COMT gene.

In some embodiments, the presently disclosed subject matter also provides methods for detecting susceptibility to dopamine partial agonist therapy in subjects suffering from or at risk for developing disorders associated with dopamine receptor activity. In some embodiments, the methods comprise obtaining a biological sample from the subject and performing or having performed one or more genotyping assays on a nucleic acid sample isolated from the subject to determine the subject's genotype with respect to a variable number tandem repeats (VNTR) polymorphism in a dopamine transporter DAT1/SLC6A3 gene, an rs4680 polymorphism in a DA-catabolizing enzyme catechol-O-methyltransferase (COMT) gene, an rs1076560 polymorphism in a D2 receptor (DRD2) gene, a 48-base-pair VNTR polymorphism in a D4 receptor (DRD4) gene, and/or an rs1800497 polymorphism in an ankyrin repeat and kinase domain containing 1 (ANKK1) gene, wherein detection of at least one allele for 9 tandem repeats of the DAT1/SLC6A3 VNTR or or four or more of a 9 tandem repeat allele of the DAT1/SLC6A3 VNTR; a COMT A allele of the rs4680 polymorphism; a 48-base-pair VNTR in DRD4 exon 3 allele; and a DRD2 T allele of the rs1076560 polymorphism, an ANKK1 TaqA1 A allele of the rs1800497 polymorphism, or both indicates that the subject is susceptible to a dopamine partial agonist therapy. In some embodiments, the dopamine partial agonist is selected from the group consisting of aripiprazole, brexipiprizole, and cariprazine. In some embodiments, at least one of the one or more genotyping assays comprises a nucleic acid amplification process followed by sequencing or gel electrophoresis of an amplification product produced thereby. In some embodiments, the one or more genotyping assays determine the subject's genotype with respect to the VNTR polymorphism in the dopamine transporter DAT1/SLC6A3 gene, the rs1076560 polymorphism in the DRD2 gene, and the 48-base-pair VNTR polymorphism in the DRD4 gene. In some embodiments, the one or more genotyping assays further comprise a genotyping assay that determines the subject's genome with respect to the rs4680 polymorphism in the COMT gene.

In some embodiments, the presently disclosed subject matter also provides methods for identifying human subjects having susceptibility to dopamine partial agonist therapies for a disorder associated with dopamine receptor activity and treating the human subjects for the disorder. In some embodiments, the methods comprise obtaining a nucleic acid sample from a human subject; performing or having performed one or more genotyping assays on a nucleic acid sample isolated from the subject to determine the subject's genotype with respect to a variable number tandem repeats (VNTR) polymorphism in a dopamine transporter DAT1/SLC6A3 gene, an rs4680 polymorphism in a DA-catabolizing enzyme catechol-O-methyltransferase (COMT) gene, an rs1076560 polymorphism in a D2 receptor (DRD2) gene, a 48-base-pair VNTR polymorphism in a D4 receptor (DRD4) gene, and/or an rs1800497 polymorphism in an ankyrin repeat and kinase domain containing 1 (ANKK1) gene; and administering a dopamine partial agonist to the subject if the one or more genotyping assays indicates that subject's genotype includes at least one allele for 9 tandem repeats of the DAT1/SLC6A3 VNTR or or four or more of a 9 tandem repeat allele of the DAT1/SLC6A3 VNTR; a COMT A allele of the rs4680 polymorphism; a 48-base-pair VNTR in DRD4 exon 3 allele; and a DRD2 T allele of the rs1076560 polymorphism, an ANKK1 TaqA1 A allele of the rs1800497 polymorphism, or both. In some embodiments, the one or more genotyping assays are performed after initiating a dopamine partial agonist therapy, and further wherein if the subject is homozygous for a VNTR 10 tandem repeat allele, the dopamine partial agonist therapy is discontinued. In some embodiments, the dopamine partial agonist is selected from the group consisting of aripiprazole, brexipiprizole, and cariprazine. In some embodiments, at least one of the genotyping assays comprises a nucleic acid amplification process followed by sequencing or gel electrophoresis of a resulting nucleic acid amplification product. In some embodiments, the one or more genotyping assays determine the subject's genotype with respect to the VNTR polymorphism in the dopamine transporter DAT1/SLC6A3 gene, the rs1076560 polymorphism in the DRD2 gene, and the 48-base-pair VNTR polymorphism in the DRD4 gene. In some embodiments, the one or more genotyping assays further comprise a genotyping assay that determines the subject's genome with respect to the rs4680 polymorphism in the COMT gene.

Thus, it is an object of the presently disclosed subject matter to provide methods for identifying human subjects having susceptibility to dopamine partial agonist therapies for a disorder associated with dopamine receptor activity and treating the human subjects for the disorder.

An object of the presently disclosed subject matter having been stated hereinabove, and which is achieved in whole or in part by the compositions and methods disclosed herein, other objects will become evident as the description proceeds when taken in connection with the accompanying Figures as best described herein below.

### BRIEF DESCRIPTION OF THE FIGURES

The accompanying drawings, which are incorporated herein by reference and constitute a part of this specification, illustrate several representative embodiments of the presently disclosed subject matter and together with the description illustrate the disclosed compositions and methods.

FIGS. 1A and 1B are bar graphs showing the effects of medication group and DAT1/SLC6A3 VNTR genotype on alcohol cue-elicited ventral striatal activation (FIG. 1A) and bar-lab drinking (FIG. 1B). These factors significantly interacted in their effects on both outcomes, such that aripiprazole (APZ; black bars), relative to placebo (PLA; white bars), reduced cue-elicited activation, and bar-lab drinking among 9R carriers but not among 10R homozygotes. FIGS. 1A and 1B are estimated marginal means±standard errors, and are adjusted for baseline drinks per day. *p<0.05 for interaction between medication and genotype; **p≤0.001 for simple effect of medication among 9R carriers.

FIGS. 2A and 2B are bar graphs showing the effects of medication group and the DA-related genetic composite measure on alcohol cue-elicited ventral striatal activation (FIG. 2A) and bar-lab drinking (FIG. 2A). These factors significantly interacted in their effects on both outcomes, such that aripiprazole (APZ; black bars), relative to placebo (PLA; white bars), reduced cue-elicited activation, and bar-lab drinking more among subjects who carried a greater number of alleles associated with higher DA. FIGS. 2A and 2B are estimated marginal means±standard errors, and are adjusted for baseline drinks per day. *p<0.05 for interaction between medication and linear effect of number of higher DA alleles; **p≤0.001 for simple effect of medication among individuals with four (4) or more higher DA alleles.

### BRIEF DESCRIPTION OF THE SEQUENCE LISTING

SEQ ID NOs: 1 and 2 are the nucleotide sequences of an oligonucleotide pair that together can be employed for determining the number of DAT1/SLC6A3 VNTR repeats in a nucleic acid sample.

SEQ ID NOs: 3 and 4 are the nucleotide sequences of an oligonucleotide pair that together can be employed for assaying DRD4 gene sequences in a nucleic acid sample.

SEQ ID NO: 5 is the sequence of the 40-base-pair variable number tandem repeat (VNTR) polymorphism. It corresponds to the insertion/deletion (indel) variation single nucleotide polymorphism (SNP) rs28363170 of the human DAT1/SLC6A3 VNTR, where the 9-repeat (9R) allele has nine consecutive repeats of SEQ ID NO: 5 and the 10-repeat (10R) allele has ten consecutive repeats of SEQ ID NO: 5.

SEQ ID NO: 6 is the nucleotide sequence of the single nucleotide polymorphism (SNP) rs1800497 in the human ANKK1 gene. The polymorphism is located at nucleotide 26 of SEQ ID NO: 6, wherein in some embodiments the nucleotide at this position is a thymine/uracil and in some embodiments is a cytosine.

SEQ ID NO: 7 is the nucleotide sequence of the single nucleotide polymorphism (SNP) rs4680 of the human COMT gene. The polymorphism is located at nucleotide 26 of SEQ ID NO: 7, wherein in some embodiments the nucleotide at this position is an adenine and in some embodiments is a guanine.

SEQ ID NO: 8 is the nucleotide sequence of the single nucleotide polymorphism (SNP) rs1076560 of the human DRD2 gene. The polymorphism is located at nucleotide 26 of SEQ ID NO: 8, wherein in some embodiments the nucleotide at this position is an adenine and in some embodiments is a cytosine.

SEQ ID NOs: 9 and 10 are nucleotide and amino acid sequences, respectively, of exemplary human DAT1/SLC6A3 gene products. The nucleotide sequence corresponds to Accession No. NM_001044.4 in the GENBANK® biosequence database, and the amino acid sequence corresponds to Accession No. NP_001035.1 in the GENBANK® biosequence database.

SEQ ID NOs: 11 and 12 are nucleotide and amino acid sequences, respectively, of exemplary human COMT gene products. The nucleotide sequence corresponds to Accession No. NM_000754.3 in the GENBANK® biosequence database, and the amino acid sequence corresponds to Accession No. NP_000745.1 in the GENBANK® biosequence database. SEQ ID NO: 7 is a subsequence of SEQ ID NO: 11 (nucleotides 696-746), and the SNP corresponds to nucleotide 721 of SEQ ID NO: 11. When nucleotide 721 of SEQ ID NO: 11 is an adenine (referred to herein as the “COMT A allele”), amino acid 158 of SEQ ID NO: 12 is a methionine. When nucleotide 721 of SEQ ID NO: 11 is a guanine (referred to herein as the “COMT G allele”), amino acid 158 of SEQ ID NO: 12 is a valine.

SEQ ID NOs: 13 and 14 are nucleotide and amino acid sequences, respectively, of exemplary human DRD1 gene products. The nucleotide sequence corresponds to Accession No. NM_000794.4 in the GENBANK® biosequence database, and the amino acid sequence corresponds to Accession No. NP_000785.1 in the GENBANK® biosequence database.

SEQ ID NOs: 15 and 16 are nucleotide and amino acid sequences, respectively, of exemplary human DRD2 gene products. The nucleotide sequence corresponds to Accession No. NM_000795.3 in the GENBANK® biosequence database, and the amino acid sequence corresponds to Accession No. NP_000786.1 in the GENBANK® biosequence database. SEQ ID NO: 8 is located in an intron region of the exemplary human DRD2 gene product represented by SEQ ID NOs: 15 and 16 and on the opposite strand as the open reading frame of SEQ ID NO: 15. Thus, when nucleotide 26 of SEQ ID NO: 8 is an adenine, the allele is referred to herein as the “DRD2 T allele”. When nucleotide 26 of SEQ ID NO: 8 is a cytosine, the allele is referred to herein as the “DRD2 G allele”.

SEQ ID NOs: 17 and 18 are nucleotide and amino acid sequences, respectively, of exemplary human DRD3 gene products. The nucleotide sequence corresponds to Accession No. NM_000796.5 in the GENBANK® biosequence database, and the amino acid sequence corresponds to Accession No. NP_000787.2 in the GENBANK® biosequence database.

SEQ ID NOs: 19 and 20 are nucleotide and amino acid sequences, respectively, of exemplary human DRD4 gene products. The nucleotide sequence corresponds to Accession No. NM_000797.3 in the GENBANK® biosequence database, and the amino acid sequence corresponds to Accession No. NP_000788.2 in the GENBANK® biosequence database.

SEQ ID NOs: 21 and 22 are nucleotide and amino acid sequences, respectively, of exemplary human ANKK1 gene products. The nucleotide sequence corresponds to Accession No. NM_178510.1 in the GENBANK® biosequence database, and the amino acid sequence corresponds to Accession No. NP_848605.1 in the GENBANK® biosequence database. SEQ ID NO: 6 is the reverse complement of a subsequence of SEQ ID NO: 21 (nucleotides 2206-2255), and the SNP corresponds to nucleotide 2231 of SEQ ID NO: 21. When nucleotide 2231 of SEQ ID NO: 21 is a guanine (referred to herein as the “ANKK1 Taq1 G allele”), amino acid 713 of SEQ ID NO: 22 is a glutamic acid. When nucleotide 2231 of SEQ ID NO: 21 is an adenine (referred to herein as the “ANKK1 Taq1 A allele”), amino acid 713 of SEQ ID NO: 22 is a lysine.

SEQ ID NO: 23 is the nucleotide sequence of an exemplary oligonucleotide that can be employed with SEQ ID NO: 2 in place of SEQ ID NO: 1 for determining the number of DAT1/SLC6A3 VNTR repeats in a nucleic acid sample.

### DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### I. Definitions

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

While the following terms are believed to be well understood by one of ordinary skill in the art, the following definitions are set forth to facilitate explanation of the presently disclosed subject matter.

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, some embodiments includes from the one particular value and/or to the other particular value. Similarly, when values are expressed as approximations, by use of the antecedent “about,” it will be understood that the particular value forms an embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed, then “less than or equal to 10” as well as “greater than or equal to 10” are also disclosed. It is also understood that the throughout the application, data are provided in a number of different formats, and that these data represent in some embodiments endpoints and starting points and in some embodiments ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

The term “and/or”, when used in the context of a list of entities, refers to the entities being present singly or in combination. Thus, for example, the phrase “at least one allele for 9 tandem repeats of the VNTR, at least one COMT A allele of the rs4680 polymorphism, at least one DRD2 T allele of the rs1076560 polymorphism, at least one a 48-base-pair VNTR in DRD4 exon 3 allele, and/or at least one ANKK1 TaqA1 A allele of the rs1800497 polymorphism” includes at least one allele for 9 tandem repeats of the VNTR, at least one COMT A allele of the rs4680 polymorphism, at least one DRD2 T allele of the rs1076560 polymorphism, at least one a 48-base-pair VNTR in DRD4 exon 3 allele, and at least one ANKK1 TaqA1 A allele of the rs1800497 polymorphism individually, but also includes any and all combinations and subcombinations of at least one allele for 9 tandem repeats of the VNTR, at least one COMT A allele of the rs4680 polymorphism, at least one DRD2 T allele of the rs1076560 polymorphism, at least one a 48-base-pair VNTR in DRD4 exon 3 allele, and/or at least one ANKK1 TaqA1 A allele of the rs1800497 polymorphism.

The terms “optional” and “optionally” as used herein indicate that the subsequently described event, circumstance, element, and/or method step may or may not occur and/or be present, and that the description includes instances where said event, circumstance, element, or method step occurs and/or is present as well as instances where it does not.

“Probes” are molecules capable of interacting with a target nucleic acid, typically in a sequence specific manner, for example through hybridization. The hybridization of nucleic acids is well understood in the art and discussed herein. Typically, a probe can be made from any combination of nucleotides, nucleotide derivatives, and/or analogs thereof as are available in the art.

“Primers” are a subset of probes which are capable of supporting some type of enzymatic manipulation and which can hybridize with a target nucleic acid such that the enzymatic manipulation can occur. A primer can be made from any combination of nucleotides or nucleotide derivatives or analogs available in the art which do not interfere with the enzymatic manipulation.

As used herein, the term “ANKK1” refers to an ankyrin repeat and kinase domain containing 1 (ANKK1) gene product, such as but not limited to those gene products described in Accession Nos. NM_178510.1 and NP_848605.1 in the GENBANK® biosequence database.

As used herein, the term “COMT” refers to a catechol-O-methyltransferase (COMT) gene product, such as but not limited to those gene products described in Accession Nos. NM_000754.3 and NP_000745.1 in the GENBANK® biosequence database.

As used herein, the terms “DAT1”, “SLC6A3” and “DAT1/SLC6A3” refers to a solute carrier family 6 member 3 (SLC6A3) gene product, such as but not limited to those gene products described in Accession Nos. NM_001044.4 and NP_001035.1 in the GENBANK® biosequence database. The SLC6A3 gene is also referred to as the dopamine transporter 1 (DAT1) gene.

As used herein, the term “DRD1” refers to a dopamine receptor D₁ (DRD1) gene product, such as but not limited to those gene products described in Accession Nos. NM_000794.4 and NP_000785.1 in the GENBANK® biosequence database.

As used herein, the term “DRD2” refers to a dopamine receptor D₂ (DRD2) gene product, such as but not limited to those gene products described in Accession Nos. NM_000795.3 and NP_000786.1 in the GENBANK® biosequence database.

As used herein, the term “DRD3” refers to a dopamine receptor D₃ (DRD3) gene product, such as but not limited to those gene products described in Accession Nos. NM_000796.5 and NP_000787.2 in the GENBANK® biosequence database.

As used herein, the term “DRD4” refers to a dopamine receptor D₄ (DRD4) gene product, such as but not limited to those gene products described in Accession Nos. NM_000797.3 and NP_000788.2 in the GENBANK® biosequence database.

As is known in the art, in some embodiments multiple gene products can be generated from a particular genetic locus, for example by alternative transcriptional initiation sites, alternative splicing, etc. It is understood that the GENBANK® Accession Nos. presented herein are meant to be exemplary only, and other gene products for which the nucleotide and/or amino acid sequences are not explicitly disclosed herein are also intended to be encompassed by the names of the corresponding genes. Thus, for example, transcript variants of the sequences in the Sequence Listing are also included with the definitions of the genes described herein, as are the amino acid variants encoded thereby.

II. Disorders Associated with Dopamine Receptor Activities

In some embodiments, the presently disclosed subject matter provides methods for treating subjects with disorders associated with dopamine receptor activity, detecting susceptibility of subject to treatment with a dopamine receptor modulator for disorders associated with dopamine receptor activity, and identifying and treating subjects (e.g., human subjects) having susceptibility to dopamine receptor partial antagonist and/or partial agonist therapies for disorders associated with dopamine receptor activity in the subjects.

As used herein, the phrase “disorder associated with dopamine receptor activity” refers to any disease, disorder, or condition at least one symptom of which can be improved or treated by administering to a subject in need thereof a dopamine receptor antagonist (such as but not limited to a dopamine partial antagonist) or a dopamine receptor agonist (such as but not limited to a dopamine partial agonist), depending on whether the symptom results from undesirably high dopamine receptor activity or undesirably low dopamine receptor activity. Exemplary disorders associated with dopamine receptor activity include, but are not limited to alcohol use disorders (AUD), opiate addiction and/or abuse, whether intentional or unintentional; depression, anxiety, compulsive disorders, and pain.

As used herein, the phrases “dopamine antagonist” and “dopamine receptor antagonist” refer to any agent that inhibits signaling through a dopamine receptor either directly or indirectly. Exemplary dopamine receptor antagonists are disclosed herein. Similarly, the phrase “dopamine agonist” and “dopamine receptor agonist” refer to any agent that enhances or augments signaling through a dopamine receptor either directly or indirectly. Exemplary dopamine receptor agonists are also disclosed herein.

As is known in the art, some antagonists and agonists have overlapping activities such that under different circumstances they can act as either antagonists or agonists. Exemplary such agents include partial agonists, competitive antagonists, inverse agonists, and mixed agonist/antagonists. As used herein, whether a given agent acts as an antagonist or an agonist can depend on the disorder for which use of the antagonist or agonist is desired.

Alcohol use disorder (AUD) is a chronic relapsing brain disease characterized by compulsive alcohol use, loss of control over alcohol intake, and a negative emotional state when not using. An estimated 16 million people have been diagnosed as having AUD in the United States alone. To be diagnosed with AUD, individuals must meet at least two of the criteria outlined in the Diagnostic and Statistical Manual of Mental Disorders (DSM) including amount or duration of consumption, inability to reduce or stop drinking, time spent drinking or recovering, craving, interference of drinking on work, school, or family, maintaining consumption despite problems resulting from consumption, reducing activities to place more emphasis on consumption, increased risk behavior while consuming or intoxicated, continued consumption despite feelings of depression or anxiety, increased average consumption over the past year, and presence of withdrawal symptoms.

Treatment for AUD can comprise counseling, behavioral modification, and pharmacological intervention. Currently, three drugs, Naltrexone, Acamprosate, and Disulfiram, are approved for treating alcohol use disorder. More recently, modulation of the dopaminergic pathway through drugs such as aripiprazole, brexipiprizole, and cariprazine has been exploited for the treatment of AUD.

Dopamine (DA) signaling regulates many aspects of AUD. Alcohol cues and intravenous alcohol self-administration both increase DA release in the human ventral striatum (VS). Relative to controls, individuals with AUD display enhanced alcohol-induced but blunted amphetamine-induced VS DA release, and, unlike controls, demonstrate no association between striatal DA release and prefrontal glucose metabolism, suggesting impaired cortical modulation of VS DA signaling.

To remediate this impairment, several dopaminergic medications have been explored as AUD treatments. Modulation of the dopaminergic pathway can occur through a variety of mechanism such as downstream modulators, dopamine receptor antagonists, and dopamine receptor agonists. For example, aripiprazole (APZ) is a high-affinity D₂ and 5-HT_{2B} partial agonist. APZ can stabilize dysregulated DA neurotransmission by increasing striatal DA synthesis among individuals with low basal synthesis capacity, and decreasing it among individuals with high basal capacity. As a result, the patient experiences a reduction in the euphoric effects of alcohol and as a consequence the cravings and desire for alcohol decrease.

However, treatment with dopamine pathway modulators such as aripiprazole, brexipiprizole, and/or cariprazine has had mixed results, with many patients not responding to the medication whereas others experienced significant improvement. A large multisite AUD clinical trial found that APZ did not significantly change the primary drinking outcome (measured as percent days abstinent), but did significantly improve other outcomes, including drinks per drinking day and an alcohol consumption biomarker.

Given APZ's effects on DA transmission, one such between-subjects difference might be DA-related genetic variation. The DA transporter (DAT) is the primary mechanism for striatal DA clearance. A 40-base-pair variable number tandem repeat (VNTR) polymorphism (rs28363170; SEQ ID NO: 5) in the 3′ untranslated region of the DAT1 gene (DAT1/SLC6A3), for which the most common allelic variants are nine (9) and ten (10) repeats, can affect DAT1 function. Relative to the 10-repeat (10R) allele, the 9-repeat (9R) allele has been associated with reduced DAT1 expression and lower striatal DAT1 availability among AUD individuals, potentially leading to relatively increased extrasynaptic DA tone. Consistent with these findings, individuals who carry the 9R allele, relative to 10R homozygotes, display greater VS activation during the anticipation and receipt of monetary reward. Further, nicotine-dependent 9R carriers display greater smoking cue-elicited VS activation and greater VS DA release after smoking.

In some embodiments, disclosed herein are methods for treating a psychiatric, mental, and/or neurological disorder (such as, for example, alcohol use disorder (AUD)). In some embodiments, the presently disclosed methods comprise assaying the nucleic acid from a subject to determine the subject's genotype with respect to the VNTR of DAT1/SLC6A3, the COMT rs4680 polymorphism, the DRD2 rs1076560 polymorphism, the DRD4 48-base-pair VNTR polymorphism, and/or the ANKK1 rs1800497 polymorphism, wherein when at least one allele for 9 tandem repeats of the VNTR, at least one COMT A allele of the rs4680 polymorphism, at least one DRD2 T allele of the rs1076560 polymorphism, at least one a 48-base-pair VNTR in DRD4 exon 3 allele; and/or at least one ANKK1 TaqA1 A allele of the rs1800497 polymorphism is detected, a dopamine modulator is administered to the subject; and wherein when two alleles for 10 tandem repeats is detected a dopamine modulator is not administered to the subject.

Thus, in some embodiments the presently disclosed methods comprise determining the subject's genotype with respect to the VNTR polymorphism in the dopamine transporter DAT1/SLC6A3 gene, the rs4680 polymorphism in the DA-catabolizing enzyme catechol-O-methyltransferase gene (COMT) gene, the rs1076560 polymorphism in the D₂ receptor (DRD2) gene, the 48-base-pair VNTR polymorphism in the D₄ receptor (DRD4) gene, and/or the rs1800497 polymorphism in the ankyrin repeat and kinase domain containing 1 (ANKK1) gene. In some embodiments, a dopamine partial agonist is administered to the subject if the one or more genotyping assays indicates that subject's genotype includes at least one allele for 9 tandem repeats of the VNTR (9R), at least one COMT A allele of the rs4680 polymorphism, at least one DRD2 T allele of the rs1076560 polymorphism, at least one a 48-base-pair VNTR in DRD4 exon 3 allele, and/or at least one ANKK1 TaqA1 A allele of the rs1800497 polymorphism. In some embodiments, the subject's genotype includes (a) at least one allele for 9 tandem repeats of the DAT1/SLC6A3 VNTR, at least one DRD2 T allele of the rs1076560 polymorphism, and at least one a 48-base-pair VNTR in DRD4 exon 3 allele; or (b) at least one allele for 9 tandem repeats of the DAT1/SLC6A3 VNTR and at least one a 48-base-pair VNTR in DRD4 exon 3 allele; or (c) at least one allele for 9 tandem repeats of the DAT1/SLC6A3 VNTR and at least one DRD2 T allele of the rs1076560 polymorphism; or (d) at least one allele for 9 tandem repeats of the DAT1/SLC6A3 VNTR; or (e) at least one allele for 9 tandem repeats of the DAT1/SLC6A3 VNTR, at least one COMT A allele of the rs4680 polymorphism, at least one DRD2 T allele of the rs1076560 polymorphism, and at least one a 48-base-pair VNTR in DRD4 exon 3 allele. In some embodiments, the subject's genotype includes (i) at least one allele for 9 tandem repeats of the DAT1/SLC6A3 VNTR, at least one COMT A allele of the rs4680 polymorphism, and at least one DRD2 T allele of the rs1076560 polymorphism; or (ii) at least one allele for 9 tandem repeats of the DAT1/SLC6A3 VNTR and at least one DRD2 T allele of the rs1076560 polymorphism; or (iii) at least one allele for 9 tandem repeats of the DAT1/SLC6A3 VNTR and at least one COMT A allele of the rs4680 polymorphism; (iv) at least one allele for 9 tandem repeats of the

DAT1/SLC6A3 VNTR, at least one COMT A allele of the rs4680 polymorphism, and at least one a 48-base-pair VNTR in DRD4 exon 3 allele; or (v) at least one allele for 9 tandem repeats of the DAT1/SLC6A3 VNTR, at least one DRD2 T allele of the rs1076560 polymorphism, and at least one a 48-base-pair VNTR in DRD4 exon 3 allele; or (vi) at least one allele for 9 tandem repeats of the DAT1/SLC6A3 VNTR, at least one COMT A allele of the rs4680 polymorphism, at least one DRD2 T allele of the rs1076560 polymorphism, and at least one a 48-base-pair VNTR in DRD4 exon 3 allele; or (vii) at least one allele for 9 tandem repeats of the DAT1/SLC6A3 VNTR; or (vii) at least one allele for 9 tandem repeats of the DAT1/SLC6A3 VNTR and at least one a 48-base-pair VNTR in DRD4 exon 3 allele.

In some embodiments, a dopamine partial agonist is not administered to the subject if the one or more genotyping assays indicates that subject's genotype includes two alleles for the DAT1 VNTR 10 tandem repeat (10R).

It is understood and herein provided that the detection of a subject's genotype with respect to the number of tandem repeats of the VNTR polymorphism of DAT1/SLC6A3, the rs4680 polymorphism in the DA-catabolizing enzyme catechol-O-methyltransferase gene (COMT) gene, the rs1076560 polymorphism in the D₂ receptor (DRD2) gene, the 48-base-pair VNTR polymorphism in the D₄ receptor (DRD4) gene, and/or the rs1800497 polymorphism in the ankyrin repeat and kinase domain containing 1 (ANKK1) gene can also be used to detect susceptibility to dopamine modulator therapy. Accordingly, disclosed herein in some embodiments are methods for detecting susceptibility to dopamine modulator therapy for a psychiatric, mental, and/or neurological disorder (such as, for example, alcohol use disorder (AUD)). In some embodiments, the presently disclosed methods comprise obtaining a biological sample from a subject, assaying nucleic acid from the biological sample from the subject to determine the subject's genotype with respect to a variable number tandem repeats (VNTR) polymorphism in a dopamine transporter gene DAT1/SLC6A3, an rs4680 polymorphism in a DA-catabolizing enzyme catechol-O-methyltransferase gene (COMT), an rs1076560 polymorphism in a D₂ receptor gene (DRD2), a 48-base-pair VNTR polymorphism in a D₄ receptor gene (DRD4), and/or an rs1800497 polymorphism in an ankyrin repeat and kinase domain containing 1 (ANKK1) gene, wherein detection of at least one allele for 9 tandem repeats of the VNTR (9R), at least one COMT A allele of the rs4680 polymorphism, at least one DRD2 T allele of the rs1076560 polymorphism, at least one a 48-base-pair VNTR in DRD4 exon 3 allele, and/or at least one ANKK1 TaqA1 A allele of the rs1800497 polymorphism indicates that the subject is susceptible to dopamine modulator therapy. In some embodiments, detection of two alleles for 10 tandem repeat polymorphism of the DAT1 VNTR (10R) indicates that the subject is not susceptible to dopamine modulator therapy.

As used in the methods for treating a psychiatric, mental, and/or neurological disorder (such as, for example, alcohol use disorder (AUD)) or methods for detecting susceptibility to dopamine treatment disclosed herein, a dopamine modulator as used in the disclosed methods can comprise any antibody, biologic, small molecule, and/or nucleic acid modulators (including, but not limited to small interfering RNAs (siRNAs), small hairpin RNAs (shRNAs), antisense molecules, zinc finger nucleases, meganucleases, TAL (TALE) nucleases, triplexes, modified triplexes). Such modulators can include agonists (including, but not limited to apomorphine, bromocriptine, cabergoline, ciladopa, dihydrexine, dinapsoline, doxathrine, epicriptine, lisuride, perfolide, piribedil, pramipexole, propylnorapomorphine, quinagolide, ropinirole, rotigotine, roxindole, and/or sumanirole), partial agonists (including, but not limited to aripiprazole, brexpiprazole, cariprazine, phencyclidine, LY-404.039, cannabidiol, quinpirole, and/or salvinorin A) indirect agonists (amphetamine, dectroamphetamine, lisdexamfetamine, and/or methylphiniate), dopamine reuptake inhibitors (including, but not limited to altropane, amfonelic acid, amineptine, benocyclidine, bupropion, 1-(3-Chlorophenyl)-4-(2-phenylethyl)piperazine (3C-PEP), difluoropine, DBL-583, GBR-12783, GBR-12935, GBR-13069, GBR-13098 GYKI-52895, Iometopane, methylphenidate, ethylphenidate, modafinil, armodafinil, (−)-3β-(4-iodophenyl)tropane-2β-pyrrolidine carboxamide (RTI-4229-229), vortioxetine, and/or vanoxerine), antagonists (including, but not limited to ALKS 3831, acepromazine, amisulpride, amoxapine, asenapine, AVN-211, azaperone, benperidol, bromopride, butaclamol, clomipramine, clozapine, chlorpromazine, chlorprothixene, clopenthixol, domperidone, droperidol, eticlopride, flupenthixol, fluphenazine, fluspirilene, haloperidol, hydroxyzine, iloperidone, iodobenzamide, ITI-007, levomepromazine, loxapine, Lu AF35700, lurasidone, mesridazine, metoclorpramide, MIN-101, nefadotride, nemonapride, olanzapine, olanzapine pamoate, paliperidone, paliperidone palmitate, penfluridol, perazine, perphenazine, pimavanserin, pimozide, prochlorperazine, promazine, quetiapine, raclopride, remoxipride, risperidone (including extended release formulations such as, for example, RBP-7000 and riperidone ISM), spiperone, spirozatrine, stepholidine, sulpiride, sultopride, tetrahydropalmatine, thiethylperazine, thioridazine, thiothixene, tiapride, trifluoperazine, trifluperidol, triflupromazine, and/or ziprasidone), regulators, and stabilizers of dopamine expression or activity including modulators that act on dopamine receptors (such as, for example, dopamine receptor D₁, dopamine receptor D₂, dopamine receptor D₃, and/or dopamine receptor D₄). For example, the dopamine modulator for use in the disclosed methods can be a dopamine partial agonist such as aripiprazole (including all formulations thereof including, but not limited to extended release formulations such as aripiprazole lauroxil; ARISTADA® brand, Alkermes, Dublin, Ireland), brexpiprazole, and/or cariprazine.

In some embodiments, it is understood and herein provided that the disclosed treatment methods can be applied prior to any dopamine modulating therapy and/or as a modification of a dopamine modulating therapy. Thus, in some embodiments, disclosed herein are methods for treating a psychiatric, mental, and/or neurological disorder (such as, for example, alcohol use disorder (AUD)) comprising assaying the nucleic acid from a subject to determine the subject's genotype with respect to a variable number tandem repeats (VNTR) polymorphism in a dopamine transporter gene DAT1/SLC6A3, an rs4680 polymorphism in a DA-catabolizing enzyme catechol-O-methyltransferase gene (COMT), an rs1076560 polymorphism in a D₂ receptor gene (DRD2), a 48-base-pair VNTR polymorphism in a D₄ receptor gene (DRD4), and/or an rs1800497 polymorphism in an ankyrin repeat and kinase domain containing 1 (ANKK1) gene, wherein the genotype of the subject with respect to the VNTR of DAT1/SLC6A3, the COMT rs4680 polymorphism, the DRD2 rs1076560 polymorphism, the DRD4 48-base-pair VNTR polymorphism, and/or the ANKK1 rs1800497 polymorphism is assayed prior to administering a dopamine modulator. Also disclosed are methods for treating a psychiatric, mental, and/or neurological disorder (such as, for example, alcohol use disorder (AUD)), wherein the genotype of the subject with respect to one or more of these genes and/or polymorphisms is determined after dopamine modulator therapy has commenced, and wherein when the subject's genome encodes two alleles for 10 tandem repeats of the DAT1/SLC6A3 VNTR, the dopamine modulator therapy is discontinued.

It is understood and herein provided that the disclosed methods for treating, methods for detecting the susceptibility to dopamine modulator therapy and kits are not limited to alcohol use disorder, but can be used for any psychiatric, mental, and/or neurological disorder where dopamine modulation can have an effect on the treatment of the subject. For example, the psychiatric, mental, and/or neurological disorder can comprise schizophrenia, bipolar disorder, depression, chemical addition (including, but not limited to cocaine addiction, opioid addiction, amphetamine (including methamphetamine) addiction, nicotine addiction, prescription drug addiction, alcohol use disorder, and Parkinson's disease. Additionally, dopamine modulator therapy can be used to modulate the severity of insomnia and/or irritability.

The presently disclosed subject matter thus provides methods for detection and treatment of conditions associated with dopamine receptor activity based on identification of nucleic acid polymorphisms of the VNTR of DAT1/SLC6A3, the COMT rs4680 polymorphism, the DRD2 rs1076560 polymorphism, the DRD4 48-base-pair VNTR polymorphism, and/or the ANKK1 rs1800497 polymorphism. Such polymorphisms can be detected using any method known in the art for detection and/or sequence identified of nucleic acids.

### III. DNA Detection and Quantification

As indicated throughout, the methods disclosed herein relate in some embodiments to the detection of nucleic acid variation(s) in the form of, for example, the alleles for the number of variable number tandem repeats (VNTR) of DAT1/SLC6A3, the COMT rs4680 polymorphism, the DRD2 rs1076560 polymorphism, the DRD4 48-base-pair VNTR polymorphism, and/or the ANKK1 rs1800497 polymorphism. For these latter expression level detections, in some embodiments the methods can comprise detecting either the abundance or presence of mRNA, or both. Alternatively, detection can in some embodiments be directed to the abundance or presence of DNA, for example, genomic DNA (gDNA) and/or complementary DNA (cDNA). Thus, in some embodiments the presently disclosed subject matter relates to methods for treating a psychiatric, mental, and/or neurological disorder (such as, for example, alcohol use disorder (AUD)) comprising assaying nucleic acid isolated from a subject to determine the genotype of the subject with respect to a variable number tandem repeat (VNTR) polymorphism in the dopamine transporter gene DAT1/SLC6A3, the COMT rs4680 polymorphism, the DRD2 rs1076560 polymorphism, the DRD4 48-base-pair VNTR polymorphism, and/or the ANKK1 rs1800497 polymorphism, wherein when at least one allele for 9 tandem repeats of the VNTR, at least one COMT A allele of the rs4680 polymorphism, at least one DRD2 T allele of the rs1076560 polymorphism, at least one a 48-base-pair VNTR in DRD4 exon 3 allele, and/or at least one ANKK1 TaqA1 A allele of the rs1800497 polymorphism is detected, a dopamine modulator is administered to the subject; and wherein when two alleles for the 10 tandem repeat embodiment is detected, a dopamine modulator is not administered to the subject. In some embodiments, the genotype of the subject with respect to the VNTR polymorphism, the COMT rs4680 polymorphism, the DRD2 rs1076560 polymorphism, the DRD4 48-base-pair VNTR polymorphism, and/or the ANKK1 rs1800497 polymorphism is determined by a nucleic acid amplification process followed by sequencing of an amplification product produced thereby, gel electrophoresis, or by direct sequencing of the subject's nucleic acids.

Also disclosed herein in some embodiments are methods for detecting susceptibility to dopamine modulator therapy for a psychiatric, mental, and/or neurological disorder (such as, for example, alcohol use disorder (AUD)) comprising obtaining a biological sample from a subject, assaying nucleic acid isolated from the biological sample from the subject to identify the genotype of the subject with respect to the variable number tandem repeats (VNTR) polymorphism in the dopamine transporter gene DAT1/SLC6A3, the COMT rs4680 polymorphism, the DRD2 rs1076560 polymorphism, the DRD4 48-base-pair VNTR polymorphism, and/or the ANKK1 rs1800497 polymorphism, wherein detection of at least one allele for 9 tandem repeats of the VNTR, at least one COMT A allele of the rs4680 polymorphism, at least one DRD2 T allele of the rs1076560 polymorphism, at least one a 48-base-pair VNTR in DRD4 exon 3 allele, and/or at least one ANKK1 TaqA1 A allele of the rs1800497 polymorphism indicates that the subject is susceptible to dopamine modulator therapy. In some embodiments, the genotype of the subject with respect to the VNTR polymorphism, the COMT rs4680 polymorphism, the DRD2 rs1076560 polymorphism, the DRD4 48-base-pair VNTR polymorphism, and/or the ANKK1 rs1800497 polymorphism is detected by a nucleic acid amplification process followed by sequencing or gel electrophoresis of the amplification product(s) obtained or by direct sequencing of nucleic acids isolated from the subject or derived therefrom (e.g., direct sequencing of a cDNA produced from mRNA isolated from the subject).

A number of widely used procedures exist for detecting and determining the sequence and/or abundance of a particular nucleic acid (e.g., DNA) in a sample. For example, the technology of PCR permits amplification and subsequent detection of minute quantities of a target nucleic acid. Details of PCR are well described in the art, including, for example, U.S. Pat. No. 4,683,195 to Mullis et al., U.S. Pat. No. 4,683,202 to Mullis, and U.S. Pat. No. 4,965,188 to Mullis et al. Generally, oligonucleotide primers are annealed to the denatured strands of a target nucleic acid, and primer extension products are formed by the polymerization of deoxynucleoside triphosphates by a polymerase. A typical PCR method involves repetitive cycles of template nucleic acid denaturation, primer annealing and extension of the annealed primers by the action of a thermostable polymerase. The process results in exponential amplification of the target nucleic acid, and thus allows the detection of targets existing in very low concentrations in a sample. It is understood and herein provided that there are variant PCR methods known in the art that may also be utilized in the disclosed methods, for example, Quantitative PCR (QPCR); microarrays, real-time PCT; hot start PCR; nested PCR; allele-specific PCR; digital droplet PCR (ddPCR), digital droplet quantitative PCR (ddQPCR), and Touchdown PCR.

### III.A. Microarrays

An array is an orderly arrangement of samples, providing a medium for matching known and unknown DNA samples based on base-pairing rules and automating the process of identifying the unknowns. An array experiment can make use of common assay systems such as microplates or standard blotting membranes, and can be created by hand or make use of robotics to deposit the sample. In general, arrays are described as macroarrays or microarrays, the difference being the size of the sample spots. Macroarrays contain sample spot sizes of about 300 microns or larger and can be easily imaged by existing gel and blot scanners. The sample spot sizes in microarray can be 300 microns or less, but typically less than 200 microns in diameter and these arrays usually contains thousands of spots. Microarrays require specialized robotics and/or imaging equipment that generally are not commercially available as a complete system. Terminologies that have been used in the literature to describe this technology include, but not limited to: biochip, DNA chip, DNA microarray, GENECHIP® brand (Affymetrix, Inc. which refers to its high density, oligonucleotide-based DNA arrays), and gene array.

DNA microarrays or DNA chips are fabricated by high-speed robotics, generally on glass or nylon substrates, for which probes with known identity are used to determine complementary binding, thus allowing massively parallel gene expression and gene discovery studies. An experiment with a single DNA chip can provide information on thousands of genes simultaneously. It is herein provided that the disclosed microarrays can be used to monitor gene expression, disease diagnosis, gene discovery, drug discovery (pharmacogenomics), and toxicological research or toxicogenomics.

There are two variants of the DNA microarray technology, in terms of the property of arrayed DNA sequence with known identity. Type I microarrays comprise a probe cDNA (typically about 500-5,000 bases long) that is immobilized to a solid surface such as glass using robot spotting and exposed to a set of targets either separately or in a mixture. This method is traditionally referred to as DNA microarray. With Type I microarrays, localized multiple copies of one or more polynucleotide sequences, preferably copies of a single polynucleotide sequence are immobilized on a plurality of defined regions of the substrate's surface. A polynucleotide refers to a chain of nucleotides ranging from 5 to 10,000 nucleotides. These immobilized copies of a polynucleotide sequence are suitable for use as probes in hybridization experiments.

To prepare beads coated with immobilized probes, beads are immersed in a solution containing the desired probe sequence and then immobilized on the beads by covalent or noncovalent means. Alternatively, when the probes are immobilized on rods, a given probe can be spotted at defined regions of the rod. Typical dispensers include a micropipette delivering solution to the substrate with a robotic system to control the position of the micropipette with respect to the substrate. There can be a multiplicity of dispensers so that reagents can be delivered to the reaction regions simultaneously. In one embodiment, a microarray is formed by using ink-jet technology based on the piezoelectric effect, whereby a narrow tube containing a liquid of interest, such as oligonucleotide synthesis reagents, is encircled by an adapter. An electric charge sent across the adapter causes the adapter to expand at a different rate than the tube and forces a small drop of liquid onto a substrate.

Tissue samples may be any sample containing polynucleotides (polynucleotide targets) of interest and obtained from any bodily fluid (blood, urine, saliva, phlegm, gastric juices, etc.), cultured cells, biopsies, or other tissue preparations. DNA or RNA can be isolated from the sample according to any of a number of methods well known to those of skill in the art. In one embodiment, total RNA is isolated using the TRIZOL™ brand total RNA isolation reagent (Life Technologies, Inc., Rockville, Md., United States of America) and RNA is isolated using oligo d(T) column chromatography or glass beads. After hybridization and processing, the hybridization signals obtained should reflect accurately the amounts of control target polynucleotide added to the sample.

The plurality of defined regions on the substrate can be arranged in a variety of formats. For example, the regions may be arranged perpendicular or in parallel to the length of the casing. Furthermore, the targets do not have to be directly bound to the substrate, but rather can be bound to the substrate through a linker group. The linker groups may typically vary from about 6 to 50 atoms long. Linker groups include ethylene glycol oligomers, diamines, diacids and the like. Reactive groups on the substrate surface react with one of the terminal portions of the linker to bind the linker to the substrate. The other terminal portion of the linker is then functionalized for binding the probes.

Sample polynucleotides may be labeled with one or more labeling moieties to allow for detection of hybridized probe/target polynucleotide complexes. The labeling moieties can include compositions that can be detected by spectroscopic, photochemical, biochemical, bioelectronic, immunochemical, electrical, optical or chemical means. The labeling moieties include radioisotopes, such as ³²P, ³³P or ³⁵S, chemiluminescent compounds, labeled binding proteins, heavy metal atoms, spectroscopic markers, such as fluorescent markers and dyes, magnetic labels, linked enzymes, mass spectrometry tags, spin labels, electron transfer donors and acceptors, biotin, and the like.

Labeling can be carried out during an amplification reaction, such as polymerase chain reaction and in vitro or in vivo transcription reactions. Alternatively, the labeling moiety can be incorporated after hybridization once a probe-target complex his formed. In one embodiment, biotin is first incorporated during an amplification step as described above. After the hybridization reaction, unbound nucleic acids are rinsed away so that the only biotin remaining bound to the substrate is that attached to target polynucleotides that are hybridized to the polynucleotide probes. Then, an avidin-conjugated fluorophore, such as avidin-phycoerythrin, that binds with high affinity to biotin is added.

Hybridization causes a polynucleotide probe and a complementary target to form a stable duplex through base pairing. Hybridization methods are well known to those skilled in the art Stringent conditions for hybridization can be defined by salt concentration, temperature, and other chemicals and conditions. Varying additional parameters, such as hybridization time, the concentration of detergent (sodium dodecyl sulfate, SDS) or solvent (formamide), and the inclusion or exclusion of carrier DNA, are well known to those skilled in the art. Additional variations on these conditions will be readily apparent to those skilled in the art.

Methods for detecting complex formation are well known to those skilled in the art. In one embodiment, the polynucleotide probes are labeled with a fluorescent label and measurement of levels and patterns of complex formation is accomplished by fluorescence microscopy, preferably confocal fluorescence microscopy. An argon ion laser excites the fluorescent label, emissions are directed to a photomultiplier and the amount of emitted light detected and quantitated. The detected signal should be proportional to the amount of probe/target polynucleotide complex at each position of the microarray. The fluorescence microscope can be associated with a computer-driven scanner device to generate a quantitative two-dimensional image of hybridization intensities. The scanned image is examined to determine the abundance/expression level of each hybridized target polynucleotide.

In a differential hybridization experiment, polynucleotide targets from two or more different biological samples are labeled with two or more different fluorescent labels with different emission wavelengths. Fluorescent signals are detected separately with different photomultipliers set to detect specific wavelengths. The relative abundances/expression levels of the target polynucleotides in two or more samples is obtained. Typically, microarray fluorescence intensities can be normalized to take into account variations in hybridization intensities when more than one microarray is used under similar test conditions. In some embodiments, individual polynucleotide probe/target complex hybridization intensities are normalized using the intensities derived from internal normalization controls contained on each microarray.

Type II microarrays comprise an array of oligonucleotides (typically about 20-80-mer oligos) or peptide nucleic acid (PNA) probes that is synthesized either in situ (on-chip) or by conventional synthesis followed by on-chip immobilization. The array is exposed to labeled sample DNA, hybridized, and the identity/abundance of complementary sequences are determined. This method, “historically” called DNA chips, was developed at Affymetrix, Inc. (Santa Clara, Calif., United States of America), which sells its photolithographically fabricated products under the GENECHIP® trademark.

The basic concept behind the use of Type II arrays for gene expression is simple: labeled cDNA or cRNA targets derived from the mRNA of an experimental sample are hybridized to nucleic acid probes attached to the solid support. By monitoring the amount of label associated with each DNA location, it is possible to infer the abundance of each mRNA species represented. Although hybridization has been used for decades to detect and quantify nucleic acids, the combination of the miniaturization of the technology and the large and growing amounts of sequence information, have enormously expanded the scale at which gene expression can be studied.

Microarray manufacturing can begin with a 5-inch square quartz wafer. Initially the quartz is washed to ensure uniform hydroxylation across its surface. Because quartz is naturally hydroxylated, it provides an excellent substrate for the attachment of chemicals, such as linker molecules, that are later used to position the probes on the arrays.

The wafer is placed in a bath of silane, which reacts with the hydroxyl groups of the quartz and forms a matrix of covalently linked molecules. The distance between these silane molecules determines the probes' packing density, allowing arrays to hold over 500,000 probe locations, or features, within a mere 1.28 square centimeters. Each of these features harbors millions of identical DNA molecules. The silane film provides a uniform hydroxyl density to initiate probe assembly. Linker molecules that are attached to the silane matrix provide a surface that can be spatially activated by light.

Probe synthesis occurs in parallel, resulting in the addition of an A, C, T, or G nucleotide to multiple growing chains simultaneously. To define which oligonucleotide chains will receive a nucleotide in each step, photolithographic masks, carrying 18 to 20 square micron windows that correspond to the dimensions of individual features, are placed over the coated wafer. The windows are distributed over the mask based on the desired sequence of each probe. When ultraviolet light is shone over the mask in the first step of synthesis, the exposed linkers become deprotected and are available for nucleotide coupling.

Once the desired features have been activated, a solution containing a single type of deoxynucleotide with a removable protection group is flushed over the wafer's surface. The nucleotide attaches to the activated linkers, initiating the synthesis process.

Although each position in the sequence of an oligonucleotide can be occupied by 1 of 4 nucleotides, resulting in an apparent need for 25×4, or 100, different masks per wafer, the synthesis process can be designed to significantly reduce this requirement. Algorithms that help minimize mask usage calculate how to best coordinate probe growth by adjusting synthesis rates of individual probes and identifying situations when the same mask can be used multiple times.

Some of the key elements of selection and design are common to the production of all microarrays, regardless of their intended application. Strategies to optimize probe hybridization, for example, are invariably included in the process of probe selection. Hybridization under particular pH, salt, and temperature conditions can be optimized by taking into account melting temperatures and using empirical rules that correlate with desired hybridization behaviors.

To obtain a complete picture of a gene's activity, some probes are selected from regions shared by multiple splice or polyadenylation variants. In other cases, unique probes that distinguish between variants are favored. Inter-probe distance is also factored into the selection process.

A different set of strategies is used to select probes for genotyping arrays that rely on multiple probes to interrogate individual nucleotides in a sequence. The identity of a target base can be deduced using four identical probes that vary only in the target position, each containing one of the four possible bases.

Alternatively, the presence of a consensus sequence can be tested using one or two probes representing specific alleles. To genotype heterozygous or genetically mixed samples, arrays with many probes can be created to provide redundant information, resulting in unequivocal genotyping. In addition, generic probes can be used in some applications to maximize flexibility. Some probe arrays, for example, allow the separation and analysis of individual reaction products from complex mixtures, such as those used in some protocols to identify single nucleotide polymorphisms (SNPs).

### III.B. Real-Time PCR

Real-time PCR monitors the fluorescence emitted during the reaction as an indicator of amplicon production during each PCR cycle (i.e., in real time) as opposed to the endpoint detection. The real-time progress of the reaction can be viewed in some systems. Real-time PCR does not detect the size of the amplicon and thus does not allow for differentiation between DNA and cDNA amplification; however, it is not influenced by non-specific amplification unless SYBR Green is used. Real-time PCR quantitation eliminates post-PCR processing of PCR products. This helps to increase throughput and reduce the chances of carryover contamination. Real-time PCR also offers a wide dynamic range of up to 10⁷-fold. Dynamic range of any assay determines how much target concentration can vary and still be quantified. A wide dynamic range means that a wide range of ratios of target and normalizer can be assayed with equal sensitivity and specificity. It follows that the broader the dynamic range, the more accurate the quantitation. When combined with RT-PCR, a real-time RT-PCR reaction reduces the time needed for measuring the amount of amplicon by providing for the visualization of the amplicon as the amplification process is progressing.

The real-time PCR system is based on the detection and quantitation of a fluorescent reporter. This signal increases in direct proportion to the amount of PCR product in a reaction. By recording the amount of fluorescence emission at each cycle, it is possible to monitor the PCR reaction during exponential phase where the first significant increase in the amount of PCR product correlates to the initial amount of target template. The higher the starting copy number of the nucleic acid target, the sooner a significant increase in fluorescence is observed. A significant increase in fluorescence above the baseline value measured during the 3-15 cycles can indicate the detection of accumulated PCR product.

A fixed fluorescence threshold is set significantly above the baseline that can be altered by the operator. The parameter C_{T} (threshold cycle) is defined as the cycle number at which the fluorescence emission exceeds the fixed threshold.

There are three main fluorescence-monitoring systems for DNA amplification: (1) hydrolysis probes; (2) hybridizing probes; and (3) DNA-binding agents. Hydrolysis probes include TAQMAN® brand probes (Applied Biosystems, Foster City, Calif., United States of America), molecular beacons, and scorpions. They use the fluorogenic 5′ exonuclease activity of Taq polymerase to measure the amount of target sequences in cDNA samples.

TAQMAN® brand probes are oligonucleotides longer than the primers (20-30 bases long with a Tm value of 10° C. higher) that contain a fluorescent dye usually on the 5′ base, and a quenching dye (usually tetramethylrhodamine; TAMRA) typically on the 3′ base. When irradiated, the excited fluorescent dye transfers energy to the nearby quenching dye molecule rather than fluorescing (this is called FRET: Förster or fluorescence resonance energy transfer). Thus, the close proximity of the reporter and quencher prevents emission of any fluorescence while the probe is intact. TAQMAN® brand probes are designed to anneal to an internal region of a PCR product. When the polymerase replicates a template on which a TAQMAN® brand probe is bound, its 5′ exonuclease activity cleaves the probe. This ends the activity of quencher (no FRET) and the reporter dye starts to emit fluorescence which increases in each cycle proportional to the rate of probe cleavage. Accumulation of PCR products is detected by monitoring the increase in fluorescence of the reporter dye (note that primers are not labelled). The TAQMAN® brand assay uses universal thermal cycling parameters and PCR reaction conditions. Because the cleavage occurs only if the probe hybridizes to the target, the origin of the detected fluorescence is specific amplification. The process of hybridization and cleavage does not interfere with the exponential accumulation of the product. One specific requirement for fluorogenic probes is that there be no G at the 5′ end. A ‘G’ adjacent to the reporter dye can quench reporter fluorescence even after cleavage.

Molecular beacons also contain fluorescent moieties (e.g., fluorescein amidite (FAM), TAMRA, tetrachlorofluorescein (TET), 6-carboxyl-X-rhodamine (ROX)) and quenching dyes (typically 4-(4-dimethylaminophenyl) diazenylbenzoic acid; DABCYL) at either end but they are designed to adopt a hairpin structure while free in solution to bring the fluorescent dye and the quencher in close proximity for FRET to occur. They have two arms with complementary sequences that form a very stable hybrid or stem. The close proximity of the reporter and the quencher in this hairpin configuration suppresses reporter fluorescence. When the beacon hybridises to the target during the annealing step, the reporter dye is separated from the quencher and the reporter fluoresces (FRET does not occur). Molecular beacons remain intact during PCR and must rebind to target every cycle for fluorescence emission. This will correlate to the amount of PCR product available. All real-time PCR chemistries allow detection of multiple DNA species (multiplexing) by designing each probe/beacon with a spectrally unique fluor/quench pair as long as the platform is suitable for melting curve analysis if SYBR Green is used. By multiplexing, the target(s) and endogenous control can be amplified in single tube.

With Scorpion probes (see e.g., U.S. Pat. No. 6,350,580 to Sorge, incorporated by reference in its entirety), sequence-specific priming and PCR product detection is achieved using a single oligonucleotide. The Scorpion probe maintains a stem-loop configuration in the unhybridized state. The fluorophore is attached to the 5′ end and is quenched by a moiety coupled to the 3′ end. The 3′ portion of the stem also contains sequence that is complementary to the extension product of the primer. This sequence is linked to the 5′ end of a specific primer via a non-amplifiable monomer. After extension of the Scorpion primer, the specific probe sequence is able to bind to its complement within the extended amplicon thus opening up the hairpin loop. This prevents the fluorescence from being quenched and a signal is observed.

Another alternative is the double-stranded DNA binding dye chemistry, which quantitates the amplicon production (including non-specific amplification and primer-dimer complex) by the use of a non-sequence specific fluorescent intercalating agent (SYBR-green I or ethidium bromide). It does not bind to ssDNA. SYBR green is a fluorogenic minor groove binding dye that exhibits little fluorescence when in solution but emits a strong fluorescent signal upon binding to double-stranded DNA. Disadvantages of SYBR green-based real-time PCR include the requirement for extensive optimization. Furthermore, non-specific amplifications require follow-up assays (melting point curve or dissociation analysis) for amplicon identification. The method has been used in genotyping subject with respect to the hemochromatosis (HFE) C282Y mutation (HFE-C282Y). Another controllable problem is that longer amplicons create a stronger signal (if combined with other factors, this may cause CDC camera saturation, see below). Normally SYBR green is used in singleplex reactions, however when coupled with melting point analysis, it can be used for multiplex reactions.

The threshold cycle or the C_{T} value is the cycle at which a significant increase in ΔRn is first detected (for definition of ΔRn, see below). The threshold cycle is when the system begins to detect the increase in the signal associated with an exponential growth of PCR product during the log-linear phase. This phase provides the most useful information about the reaction (certainly more important than the end-point). The slope of the log-linear phase is a reflection of the amplification efficiency. The efficiency (Eff) of the reaction can be calculated by the formula: Eff=10^{(-1/slope)}−1. The efficiency of the PCR should be 90−100% (3.6>slope>3.1). A number of variables can affect the efficiency of the PCR. These factors include length of the amplicon, secondary structure and primer quality. Although valid data can be obtained that fall outside of the efficiency range, the qRT-PCR should be further optimized or alternative amplicons designed. For the slope to be an indicator of real amplification (rather than signal drift), there has to be an inflection point. This is the point on the growth curve when the log-linear phase begins. It also represents the greatest rate of change along the growth curve. (Signal drift is characterized by gradual increase or decrease in fluorescence without amplification of the product.) The important parameter for quantitation is the C_{T}. The higher the initial amount of genomic DNA, the sooner accumulated product is detected in the PCR process, and the lower the C_{T} value. The threshold should be placed above any baseline activity and within the exponential increase phase (which looks linear in the log transformation). Some software allows determination of the cycle threshold (C_{T}) by a mathematical analysis of the growth curve. This provides better run-to-run reproducibility. A C_{T} value of 40 means no amplification and this value cannot be included in the calculations. Besides being used for quantitation, the C_{T} value can be used for qualitative analysis as a pass/fail measure.

Multiplex TAQMAN® brand assays can be performed using multiple dyes with distinct emission wavelengths. Available dyes for this purpose are FAM, TET, VIC, and the xanthene fluorophore JOE (the most expensive). TAMRA is reserved as the quencher on the probe and ROX as the passive reference. For best results, the combination of FAM (target) and VIC (endogenous control) is recommended (they have the largest difference in emission maximum) whereas JOE and VIC should not be combined. It is important that if the dye layer has not been chosen correctly, the machine will still read the other dye's spectrum. For example, both VIC and FAM emit fluorescence in a similar range to each other and when doing a single dye, the wells should be labelled correctly. In the case of multiplexing, the spectral compensation for the post run analysis should be turned on (on the ABI PRISM® 7700 brand sequence detection system of Applied Biosystems: Instrument/Diagnostics/Advanced Options/Miscellaneous). Activating spectral compensation improves dye spectral resolution.

### III.C. Nested PCR

The disclosed methods can in some embodiments further utilize nested PCR. Nested PCR increases the specificity of DNA amplification, by reducing background due to non-specific amplification of DNA. Two sets of primers are being used in two successive PCRs. In the first reaction, one pair of primers is used to generate DNA products, which besides the intended target, may still consist of non-specifically amplified DNA fragments. The product(s) are then used in a second PCR with a set of primers whose binding sites are completely or partially different from and located 3′ of each of the primers used in the first reaction. Nested PCR is often more successful in specifically amplifying long DNA fragments than conventional PCR, but it requires more detailed knowledge of the target sequences.

### III.D. Primers and Probes

As used herein, “primers” are a subset of probes which are capable of supporting some type of enzymatic manipulation and which can hybridize with a target nucleic acid such that the enzymatic manipulation can occur. A primer can be made from any combination of nucleotides or nucleotide derivatives or analogs available in the art which do not interfere with the enzymatic manipulation.

As used herein, “probes” are molecules capable of interacting with a target nucleic acid, typically in a sequence specific manner, for example through hybridization. The hybridization of nucleic acids is well understood in the art and discussed herein. Typically, a probe can be made from any combination of nucleotides or nucleotide derivatives or analogs available in the art.

Disclosed are compositions including primers and probes, which are capable of interacting with DAT1/SLC6A3, COMT, DRD2, DRD4, and/or ANKK1 nucleic acids or their complements. In some embodiments, the primers are used to support nucleic acid extension reactions, nucleic acid replication reactions, and/or nucleic acid amplification reactions. Typically, the primers are capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, and/or reverse transcription.

Techniques and conditions that amplify the primer in a sequence-specific manner are also disclosed. In some embodiments, the primers are used for DNA amplification reactions including but not limited to PCR, or for direct sequencing. It is understood that in some embodiments, the primers can also be extended using non-enzymatic techniques where, for example, the nucleotides or oligonucleotides used to extend the primer can be modified such that they chemically react to extend the primer in a sequence specific manner. Typically, the disclosed primers hybridize with the disclosed nucleic acids and/or regions of the nucleic acids and/or they hybridize with the complement of the nucleic acids and/or complement of a region of the nucleic acids. As an example of the use of primers, one or more primers can be used to create extension products from and templated by a first nucleic acid.

The size of the primers or probes for interaction with the nucleic acids can be any size that supports the desired enzymatic manipulation of the primer, such as DNA amplification or the simple hybridization of the probe or primer. In some embodiments, a typical primer or probe would be at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

In some embodiments, a primer or probe can be less than or equal to 6, 7, 8, 9, 10, 11, 12 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

The primers for the nucleic acid of interest typically will be used to produce extension products and/or other replicated or amplified products that contain a region of the nucleic acid of interest. The size of the product can be such that the size can be accurately determined to within in some embodiments 3 nucleotides, within in some embodiments 2 nucleotides, and within in some embodiments 1 nucleotide.

In some embodiments, the product can be, for example, at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

In some embodiments, the product can be, for example, less than or equal to 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

Thus, it is understood and herein provided that the disclosed RT-PCR and PCR reactions that comprise a portion of the disclosed methods and/or are performed using the disclosed kits require forward and reverse primers to form a primer pair. Herein disclosed, the forward and reverse primer pair in the disclosed methods and kits can in some embodiments be SEQ ID NO: 1 and/or SEQ ID NO: 23 in combination with SEQ ID NO: 2, and in some embodiments can be SEQ ID NO: 3 in combination with SEQ ID NO: 4.

It is understood and herein provided that there are situations where it can be advantageous to utilize more than one primer pair to detect the presence of a fusion, truncation, and/or overexpression mutation in a nucleic acid. Such RT-PCR or PCR reactions can be conducted separately or in a single reaction. When multiple primer pairs are placed into a single reaction, this is referred to as “multiplex PCR.” By way of example and not limitation, the reaction can comprise a first DAT1/SLC6A3 forward and reverse primer pair as well as a second DAT1/SLC6A3 forward and reverse primer pair, and in some embodiments third and/or additional DAT1/SLC6A3 forward and reverse primer pairs. In some embodiments, one or both members of the second (and/or subsequent) forward and reverse primer pair can be internal (i.e., nested) to one or both members of the first DAT1/SLC6A3 forward and reverse primer pair.

Thus, disclosed herein in one aspect are methods for detecting a susceptibility to dopamine modulator therapy or treating a psychiatric, mental, and/or neurological disorder (such as, for example, AUD), comprising assaying DNA and/or RNA isolated from a biological sample for the number genotype of alleles coding for the number of VNTR repeats comprising wherein the PCR reaction a primer pair capable of specifically hybridizing to one or more DAT1/SLC6A3 sequences for example, 5′-TGTGGTGTAGGGAACGGCCTGAG-3′ (SEQ ID NO: 1) and 5′-CTTCCTGGAGGTCACGGCTCAAGG-3′ (SEQ ID NO: 2). An alternative forward primer is 5′-TGCGGTGTAGGGAACGGCCTGAG-3′ (SEQ ID NO: 23). The methods can further comprise the use of primer pairs that specifically hybridize to DRD4 such as, for example, 5′-AGGACCCTCATGGCCTTG-3′ (SEQ ID NO: 3) and 5′-GCGACTACGTGGTCTACTCG-3′ (SEQ ID NO: 4).

### III.E. Fluorescent Change Probes and Primers

Fluorescent change probes and fluorescent change primers refer to all probes and primers that involve a change in fluorescence intensity or wavelength based on a change in the form or conformation of the probe or primer and nucleic acid to be detected, assayed or replicated. Examples of fluorescent change probes and primers include molecular beacons, Amplifluors, FRET probes, cleavable FRET probes, TAQMAN™ brand probes, Scorpion primers, fluorescent triplex oligos including but not limited to triplex molecular beacons or triplex FRET probes, fluorescent water-soluble conjugated polymers, PNA probes and QPNA probes.

Fluorescent change probes and primers can be classified according to their structure and/or function. Fluorescent change probes include hairpin quenched probes, cleavage quenched probes, cleavage activated probes, and fluorescent activated probes. Fluorescent change primers include stem quenched primers and hairpin quenched primers.

Hairpin quenched probes are probes that when not bound to a target sequence form a hairpin structure (and, typically, a loop) that brings a fluorescent label and a quenching moiety into proximity such that fluorescence from the label is quenched. When the probe binds to a target sequence, the stem is disrupted, the quenching moiety is no longer in proximity to the fluorescent label and fluorescence increases. Examples of hairpin quenched probes are molecular beacons, fluorescent triplex oligos, triplex molecular beacons, triplex FRET probes, and QPNA probes.

Cleavage activated probes are probes where fluorescence is increased by cleavage of the probe. Cleavage activated probes can include a fluorescent label and a quenching moiety in proximity such that fluorescence from the label is quenched. When the probe is clipped or digested (typically by the 5′-3′ exonuclease activity of a polymerase during amplification), the quenching moiety is no longer in proximity to the fluorescent label and fluorescence increases. TAQMAN® brand probes are an example of cleavage activated probes.

Cleavage quenched probes are probes where fluorescence is decreased or altered by cleavage of the probe. Cleavage quenched probes can include an acceptor fluorescent label and a donor moiety such that, when the acceptor and donor are in proximity, fluorescence resonance energy transfer from the donor to the acceptor causes the acceptor to fluoresce. The probes are thus fluorescent, for example, when hybridized to a target sequence. When the probe is clipped or digested (typically by the 5′-3′ exonuclease activity of a polymerase during amplification), the donor moiety is no longer in proximity to the acceptor fluorescent label and fluorescence from the acceptor decreases. If the donor moiety is itself a fluorescent label, it can release energy as fluorescence (typically at a different wavelength than the fluorescence of the acceptor) when not in proximity to an acceptor. The overall effect would then be a reduction of acceptor fluorescence and an increase in donor fluorescence. Donor fluorescence in the case of cleavage quenched probes is equivalent to fluorescence generated by cleavage activated probes with the acceptor being the quenching moiety and the donor being the fluorescent label. Cleavable FRET (fluorescence resonance energy transfer) probes are an example of cleavage quenched probes.

Fluorescent activated probes are probes or pairs of probes where fluorescence is increased or altered by hybridization of the probe to a target sequence. Fluorescent activated probes can include an acceptor fluorescent label and a donor moiety such that, when the acceptor and donor are in proximity (when the probes are hybridized to a target sequence), fluorescence resonance energy transfer from the donor to the acceptor causes the acceptor to fluoresce. Fluorescent activated probes are typically pairs of probes designed to hybridize to adjacent sequences such that the acceptor and donor are brought into proximity. Fluorescent activated probes can also be single probes containing both a donor and acceptor where, when the probe is not hybridized to a target sequence, the donor and acceptor are not in proximity but where the donor and acceptor are brought into proximity when the probe hybridized to a target sequence. This can be accomplished, for example, by placing the donor and acceptor on opposite ends of the probe and placing target complement sequences at each end of the probe where the target complement sequences are complementary to adjacent sequences in a target sequence. If the donor moiety of a fluorescent activated probe is itself a fluorescent label, it can release energy as fluorescence (typically at a different wavelength than the fluorescence of the acceptor) when not in proximity to an acceptor (that is, when the probes are not hybridized to the target sequence). When the probes hybridize to a target sequence, the overall effect would then be a reduction of donor fluorescence and an increase in acceptor fluorescence. FRET probes are an example of fluorescent activated probes.

Stem quenched primers are primers that when not hybridized to a complementary sequence form a stem structure (either an intramolecular stem structure or an intermolecular stem structure) that brings a fluorescent label and a quenching moiety into proximity such that fluorescence from the label is quenched. When the primer binds to a complementary sequence, the stem is disrupted, the quenching moiety is no longer in proximity to the fluorescent label and fluorescence increases. In the disclosed method, stem quenched primers are used as primers for nucleic acid synthesis and thus become incorporated into the synthesized or amplified nucleic acid. Examples of stem quenched primers are peptide nucleic acid quenched primers and hairpin quenched primers.

Peptide nucleic acid quenched primers are primers associated with a peptide nucleic acid quencher or a peptide nucleic acid fluor to form a stem structure. The primer contains a fluorescent label or a quenching moiety and is associated with either a peptide nucleic acid quencher or a peptide nucleic acid fluor, respectively. This puts the fluorescent label in proximity to the quenching moiety. When the primer is replicated, the peptide nucleic acid is displaced, thus allowing the fluorescent label to produce a fluorescent signal.

Hairpin quenched primers are primers that when not hybridized to a complementary sequence form a hairpin structure (and, typically, a loop) that brings a fluorescent label and a quenching moiety into proximity such that fluorescence from the label is quenched. When the primer binds to a complementary sequence, the stem is disrupted, the quenching moiety is no longer in proximity to the fluorescent label and fluorescence increases. Hairpin quenched primers are typically used as primers for nucleic acid synthesis and thus become incorporated into the synthesized or amplified nucleic acid. Examples of hairpin quenched primers are Amplifluor primers and Scorpion primers (see e.g., U.S. Patent Application Publication No. 2010/0144836 of Van England et al., incorporated herein by reference in its entirety).

Cleavage activated primers are similar to cleavage activated probes except that they are primers that are incorporated into replicated strands and are then subsequently cleaved.

### III.F. Labels

To aid in detection and quantitation of nucleic acids produced using the disclosed methods, labels can be directly incorporated into nucleotides and nucleic acids or can be coupled to detection molecules such as probes and primers. As used herein, a label is any molecule that can be associated with a nucleotide or nucleic acid, directly or indirectly, and which results in a measurable, detectable signal, either directly or indirectly. Many such labels for incorporation into nucleotides and nucleic acids or coupling to nucleic acid probes are known to those of skill in the art. Examples of labels suitable for use in the disclosed method are radioactive isotopes, fluorescent molecules, phosphorescent molecules, enzymes, antibodies, and ligands. Fluorescent labels, especially in the context of fluorescent change probes and primers, are useful for real-time detection of amplification.

Examples of suitable fluorescent labels include fluorescein isothiocyanate (FITC), 5,6-carboxymethyl fluorescein, Texas red, nitrobenz-2-oxa-1,3-diazol-4-yl (NBD), coumarin, dansyl chloride, rhodamine, amino-methyl coumarin (AMCA), Eosin, Erythrosin, BODIPY®, CASCADE BLUE®, OREGON GREEN®, pyrene, lissamine, xanthenes, acridines, oxazines, phycoerythrin, macrocyclic chelates of lanthanide ions such as quantum dye, fluorescent energy transfer dyes, such as thiazole orange-ethidium heterodimer, and the cyanine dyes Cy3, Cy3.5, Cy5, Cy5.5 and Cy7. Examples of other specific fluorescent labels include 3-Hydroxypyrene 5,8,10-Tri Sulfonic acid, 5-Hydroxy Tryptamine (5-HT), Acid Fuchsin, Alizarin Complexon, Alizarin Red, Allophycocyanin, Aminocoumarin, Anthroyl Stearate, Astrazon Brilliant Red 4G, Astrazon Orange R, Astrazon Red 6B, Astrazon Yellow 7 GLL, Atabrine, Auramine, Aurophosphine, Aurophosphine G, BAO 9 (Bisaminophenyloxadiazole), BCECF, Berberine Sulphate, Bisbenzamide, Blancophor FFG Solution, Blancophor SV, Bodipy Fl, Brilliant Sulphoflavin FF, Calcien Blue, Calcium Green, Calcofluor RW Solution, Calcofluor White, Calcophor White ABT Solution, Calcophor White Standard Solution, Carbostyryl, Cascade Yellow, Catecholamine, Chinacrine, Coriphosphine O, Coumarin-Phalloidin, CY3.1 8, CY5.1 8, CY7, Dans (1-Dimethyl Amino Naphaline 5 Sulphonic Acid), Dansa (Diamino Naphtyl Sulphonic Acid), Dansyl NH-CH3, Diamino Phenyl Oxydiazole (DAO), Dimethylamino-5-Sulphonic acid, Dipyrrometheneboron Difluoride, Diphenyl Brilliant Flavine 7GFF, Dopamine, Erythrosin ITC, Euchrysin, FIF (Formaldehyde Induced Fluorescence), Flazo Orange, Fluo 3, Fluorescamine, Fura-2, Genacryl Brilliant Red B, Genacryl Brilliant Yellow 10GF, Genacryl Pink 3G, Genacryl Yellow 5GF, Gloxalic Acid, Granular Blue, Haematoporphyrin, Indo-1, Intrawhite Cf Liquid, Leucophor PAF, Leucophor SF, Leucophor WS, Lissamine Rhodamine B200 (RD200), Lucifer Yellow CH, Lucifer Yellow VS, Magdala Red, Marina Blue, Maxilon Brilliant Flavin 10 GFF, Maxilon Brilliant Flavin 8 GFF, MPS (Methyl Green Pyronine Stilbene), Mithramycin, NBD Amine, Nitrobenzoxadidole, Noradrenaline, Nuclear Fast Red, Nuclear Yellow, Nylosan Brilliant Flavin EBG, Oxadiazole, Pacific Blue, Pararosaniline (Feulgen), Phorwite AR Solution, Phorwite BKL, Phorwite Rev, Phorwite RPA, Phosphine 3R, Phthalocyanine, Phycoerythrin R, Phycoerythrin B, Polyazaindacene Pontochrome Blue Black, Porphyrin, Primuline, Procion Yellow, Pyronine, Pyronine B, Pyrozal Brilliant Flavin 7GF, Quinacrine Mustard, Rhodamine 123, Rhodamine 5 GLD, Rhodamine 6G, Rhodamine B, Rhodamine B 200, Rhodamine B Extra, Rhodamine BB, Rhodamine BG, Rhodamine WT, Serotonin, Sevron Brilliant Red 2B, Sevron Brilliant Red 4G, Sevron Brilliant Red B, Sevron Orange, Sevron Yellow L, SITS (Primuline), SITS (Stilbene Isothiosulphonic acid), Stilbene, Snarf 1, sulpho Rhodamine B Can C, Sulpho Rhodamine G Extra, Tetracycline, Thiazine Red R, Thioflavin S, Thioflavin TCN, Thioflavin 5, Thiolyte, Thiozol Orange, Tinopol CBS, True Blue, Ultralite, Uranine B, Uvitex SFC, Xylene Orange, and XRITC.

The absorption and emission maxima, respectively, for some of these fluors are: FITC (490 nm; 520 nm), Cy3 (554 nm; 568 nm), Cy3.5 (581 nm; 588 nm), Cy5 (652 nm: 672 nm), Cy5.5 (682 nm; 703 nm) and Cy7 (755 nm; 778 nm), thus allowing their simultaneous detection. Other examples of fluorescein dyes include 6-carboxyfluorescein (6-FAM), 2′,4′,1,4,-tetrachlorofluorescein (TET), 2′,4′,5′,7′,1,4-hexachlorofluorescein (HEX), 2′,7′-dimethoxy-4′,5′-dichloro-6-carboxyrhodamine (JOE), 2′-chloro-5′-fluoro-7′,8′-fused phenyl-1,4-dichloro-6-carboxyfluorescein (NED), and 2′-chloro-7′-phenyl-1,4-dichloro-6-carboxyfluorescein (VIC). Fluorescent labels can be obtained from a variety of commercial sources, including but not limited to Amersham Pharmacia Biotech, Piscataway, N.J., United States of America; Molecular Probes, Eugene, Oreg., United States of America; and Research Organics, Cleveland, Ohio, United States of America.

Additional labels of interest include those that provide for signal only when the probe with which they are associated is specifically bound to a target molecule, where such labels include: “molecular beacons” as described in Tyagi & Kramer, 1996 and European Patent Publication No. 0 070 685 B 1, the disclosure of each of which is incorporated herein by reference in its entirety. Other labels of interest include those described in U.S. Pat. No. 5,563,037 to Sutherland & Patterson (incorporated herein by reference in its entirety).

Labeled nucleotides are a form of label that can be directly incorporated into the amplification products during synthesis. Examples of labels that can be incorporated into amplified nucleic acids include nucleotide analogs such as BrdUrd, aminoallyldeoxyuridine, 5-methylcytosine, bromouridine, and nucleotides modified with biotin or with suitable haptens such as digoxygenin. Suitable fluorescence-labeled nucleotides are Fluorescein-isothiocyanate-dUTP, Cyanine-3-dUTP and Cyanine-5-dUTP. One example of a nucleotide analog label for DNA is BrdUrd (bromodeoxyuridine, BrdUrd, BrdU, BUdR; Sigma-Aldrich Co., St. Louis, Mo., United States of America). Other examples of nucleotide analogs for incorporation of label into DNA are AA-dUTP (aminoallyl-deoxyuridine triphosphate, Sigma-Aldrich Co.), and 5-methyl-dCTP (Roche Molecular Biochemicals, Indianapolis, Ind., United States of America). One example of a nucleotide analog for incorporation of label into RNA is biotin-16-UTP (biotin-16-uridine-5′-triphosphate, Roche Molecular Biochemicals). Fluorescein, Cy3, and Cy5 can be linked to dUTP for direct labeling. Cy3.5 and Cy7 are available as avidin or anti-digoxygenin conjugates for secondary detection of biotin- or digoxygenin-labeled probes.

Labels that are incorporated into amplified nucleic acid, such as biotin, can be subsequently detected using sensitive methods well-known in the art. For example, biotin can be detected using streptavidin-alkaline phosphatase conjugate (Tropix, Inc., Bedford, Mass., United States of America), which is bound to the biotin and subsequently detected by chemiluminescence of suitable substrates (for example, chemiluminescent substrate CSPD: disodium, 3-(4-methoxyspiro-[1,2,-dioxetane-3-2′-(5′-chloro)tricyclo [3.3.1.1^{3,7}]decane]-4-yl) phenyl phosphate; Tropix, Inc.). Labels can also be enzymes, such as alkaline phosphatase, soybean peroxidase, horseradish peroxidase and polymerases, that can be detected, for example, with chemical signal amplification or by using a substrate to the enzyme which produces light (for example, a chemiluminescent 1,2-dioxetane substrate) or fluorescent signal.

Molecules that combine two or more of these labels are also considered labels. Any of the known labels can be used with the disclosed probes, tags, and method to label and detect nucleic acid amplified using the disclosed method. Methods for detecting and measuring signals generated by labels are also known to those of skill in the art. For example, radioactive isotopes can be detected by scintillation counting or direct visualization; fluorescent molecules can be detected with fluorescent spectrophotometers; phosphorescent molecules can be detected with a spectrophotometer or directly visualized with a camera; enzymes can be detected by detection or visualization of the product of a reaction catalyzed by the enzyme; antibodies can be detected by detecting a secondary label coupled to the antibody. As used herein, detection molecules are molecules which interact with amplified nucleic acid and to which one or more labels are coupled.

The disclosed methods can use any sequencing technique known in the art including, but not limited to methods disclosed by Sanger (dideoxy method), Maxam-Gilbert (chemical cleavage), automated sequencing (for example ABI systems) and next generation sequencing. From a technical perspective High-throughput or Next Generation Sequencing (NGS) represents an attractive option for detecting the somatic mutations within a gene. Unlike PCR, microarrays, high-resolution melting and mass spectrometry, which all indirectly infer sequence content, NGS directly ascertains the identity of each base and the order in which they fall within a gene. The newest platforms on the market have the capacity to cover an exonic region 10,000 times over, meaning the content of each base position in the sequence is measured thousands of different times. This high level of coverage ensures that the consensus sequence is extremely accurate and enables the detection of rare variants within a heterogeneous sample. For example, in a sample extracted from Formalin-fixed, Paraffin-embedded (FFPE) tissue, relevant mutations are only present at a frequency of 1% with the wild-type allele comprising the remainder. When this sample is sequenced at 10,000×coverage, even rare alleles comprising only 1% of the sample are each uniquely measured 100 times over. Thus, NGS can provide reliably accurate results with very high sensitivity, making it ideal for clinical diagnostic testing of FFPEs and other mixed samples.

Examples of Next Generation Sequencing techniques include, but are not limited to Massively Parallel Signature Sequencing (MPSS; see U.S. Pat. No. 6,013,445 to Albrecht et al., incorporated by reference herein in its entirety), Polony sequencing (see U.S. Pat. No. 9,982,296 to Edwards, incorporated by reference herein in its entirety), pyrosequencing, Reversible dye-terminator sequencing, Sequencing by Oligonucleotide Ligation and Detection (SOLiD) sequencing (Thermo Fisher Scientific), Ion semiconductor sequencing, DNA nanoball sequencing, Helioscope single molecule sequencing, Single molecule real time (SMRT) sequencing, Single molecule real time (RNAP) sequencing, and Nanopore DNA sequencing. MPSS was a bead-based method that used a complex approach of adapter ligation followed by adapter decoding, reading the sequence in increments of four nucleotides; this method made it susceptible to sequence-specific bias or loss of specific sequences. Polony sequencing, combined an in vitro paired-tag library with emulsion PCR, an automated microscope, and ligation-based sequencing chemistry to sequence an E. coli genome at an accuracy of greater than 99.9999% and a cost approximately one-tenth that of Sanger sequencing.

A parallelized version of pyrosequencing, the method amplifies DNA inside water droplets in an oil solution (emulsion PCR), with each droplet containing a single DNA template attached to a single primer-coated bead that then forms a clonal colony. The sequencing machine contains many picolitre-volume wells each containing a single bead and sequencing enzymes. Pyrosequencing uses luciferase to generate light for detection of the individual nucleotides added to the nascent DNA, and the combined data are used to generate sequence read-outs. This technology provides intermediate read length and price per base compared to Sanger sequencing on one end and Solexa (see e.g., Quake et al., 2003) and SOLiD on the other.

A sequencing technology based on reversible dye-terminators. DNA molecules are first attached to primers on a slide and amplified so that local clonal colonies are formed. Four types of reversible terminator bases (RT-bases) are added, and non-incorporated nucleotides are washed away. Unlike pyrosequencing, the DNA can only be extended one nucleotide at a time. A camera takes images of the fluorescently labeled nucleotides, then the dye along with the terminal 3′ blocker is chemically removed from the DNA, allowing the next cycle.

SOLiD technology employs sequencing by ligation. Here, a pool of all possible oligonucleotides of a fixed length are labeled according to the sequenced position. Oligonucleotides are annealed and ligated; the preferential ligation by DNA ligase for matching sequences results in a signal informative of the nucleotide at that position. Before sequencing, the DNA is amplified by emulsion PCR. The resulting bead, each containing only copies of the same DNA molecule, are deposited on a glass slide. The result is sequences of quantities and lengths comparable to Illumina sequencing.

Ion semiconductor sequencing is based on using standard sequencing chemistry, but with a novel, semiconductor based detection system. This method of sequencing is based on the detection of hydrogen ions that are released during the polymerization of DNA, as opposed to the optical methods used in other sequencing systems. A micro well containing a template DNA strand to be sequenced is flooded with a single type of nucleotide. If the introduced nucleotide is complementary to the leading template nucleotide it is incorporated into the growing complementary strand. This causes the release of a hydrogen ion that triggers a hypersensitive ion sensor, which indicates that a reaction has occurred. If homopolymer repeats are present in the template sequence multiple nucleotides will be incorporated in a single cycle. This leads to a corresponding number of released hydrogens and a proportionally higher electronic signal.

DNA nanoball sequencing is a type of high throughput sequencing technology used to determine the entire genomic sequence of an organism. The method uses rolling circle replication to amplify small fragments of genomic DNA into DNA nanoballs. Unchained sequencing by ligation is then used to determine the nucleotide sequence. This method of DNA sequencing allows large numbers of DNA nanoballs to be sequenced per run.

Helicos's single-molecule sequencing uses DNA fragments with added polyA tail adapters, which are attached to the flow cell surface (Helicos Inc., Cambridge, Mass., United States of America; see also Milos, 2010). The next steps involve extension-based sequencing with cyclic washes of the flow cell with fluorescently labeled nucleotides (one nucleotide type at a time, as with the Sanger method). The reads are performed by the HELIOSCOPE™ brand sequencer (Helicos Inc.).

SMRT sequencing (Pacific Biosciences of California, Inc., Menlo Park, Calif., United States of America) is based on the sequencing by synthesis approach (see U.S. Patent Application Publication No. 2009/0208957, incorporated herein by reference in its entirety). The DNA is synthesized in zero-mode wave-guides (ZMWs)—small well-like containers with the capturing tools located at the bottom of the well. The sequencing is performed with use of unmodified polymerase (attached to the ZMW bottom) and fluorescently labeled nucleotides flowing freely in the solution. The wells are constructed in a way that only the fluorescence occurring by the bottom of the well is detected. The fluorescent label is detached from the nucleotide at its incorporation into the DNA strand, leaving an unmodified DNA strand.

Single molecule real time sequencing based on RNA polymerase (RNAP), which is attached to a polystyrene bead, with distal end of sequenced DNA is attached to another bead, with both beads being placed in optical traps. RNAP motion during transcription brings the beads in closer and their relative distance changes, which can then be recorded at a single nucleotide resolution. The sequence is deduced based on the four readouts with lowered concentrations of each of the four nucleotide types (similarly to Sangers method).

Nanopore sequencing is based on the readout of electrical signal occurring at nucleotides passing by alpha-hemolysin pores covalently bound with cyclodextrin. The DNA passing through the nanopore changes its ion current. This change is dependent on the shape, size and length of the DNA sequence. Each type of the nucleotide blocks the ion flow through the pore for a different period of time.

VisiGen Biotechnologies, Inc. (Houston, Tex., United States of America) uses a specially engineered DNA polymerase. This polymerase acts as a sensor—having incorporated a donor fluorescent dye by its active center. This donor dye acts by FRET (fluorescent resonant energy transfer), inducing fluorescence of differently labeled nucleotides. This approach allows reads performed at the speed at which polymerase incorporates nucleotides into the sequence (several hundred per second). The nucleotide fluorochrome is released after the incorporation into the DNA strand. See U.S. Pat. No. 7,211,414 to Hardin et al., the disclosure of which is incorporated herein in its entirety.

Sequencing by hybridization is a non-enzymatic method that uses a DNA microarray. A single pool of DNA whose sequence is to be determined is fluorescently labeled and hybridized to an array containing known sequences. Strong hybridization signals from a given spot on the array identify its sequence in the DNA being sequenced.

Mass spectrometry may be used to determine mass differences between DNA fragments produced in chain-termination reactions.

Another NGS approach is sequencing by synthesis (SBS) technology which is capable of overcoming the limitations of existing pyrosequencing based NGS platforms.

Such technologies rely on complex enzymatic cascades for read out, are unreliable for the accurate determination of the number of nucleotides in homopolymeric regions and require excessive amounts of time to run individual nucleotides across growing DNA strands. The SBS NGS platform uses a direct sequencing approach to produce a sequencing strategy with very a high precision, rapid pace and low cost.

SBS sequencing is initialized by fragmenting of the template DNA into fragments, amplification, annealing of DNA sequencing primers, and finally affixing as a high-density array of spots onto a glass chip. The array of DNA fragments are sequenced by extending each fragment with modified nucleotides containing cleavable chemical moieties linked to fluorescent dyes capable of discriminating all four possible nucleotides. The array is scanned continuously by a high-resolution electronic camera (Measure) to determine the fluorescent intensity of each base (A, C, G or T) that was newly incorporated into the extended DNA fragment. After the incorporation of each modified base the array is exposed to cleavage chemistry to break off the fluorescent dye and end cap allowing additional bases to be added. The process is then repeated until the fragment is completely sequenced or maximal read length has been achieved.

### IV. Multiple Polymorphisms

In some embodiments, it is understood and herein provided that polymorphisms in other genes also affect DA tone and reward-related behavior, including AUD-specific phenotypes. Recently, multilocus genetic composite scores have been used to quantify the contributions of multiple polymorphisms in DA-related genes to reward-related phenotypes. Composites comprising genotypes at the DAT1/SLC6A3 VNTR and polymorphisms in the genes encoding the DA-catabolizing enzyme catechol-O-methyltransferase (COMT) and the D₂ and D₄ receptors (DRD2 and DRD4) have been reported to predict striatal response to reward, such that individuals who carry a larger number of alleles putatively associated with high basal DA tone display greater reward-related VS activation. The polymorphisms aggregated in these composites include single nucleotide polymorphisms (SNPs) in COMT (rs4680, also known as Va1158Met), DRD2 (rs1076560), ANKK1 (rs1800497; also known as Taq1A, and originally believed to be in the adjacent DRD2 promoter), and a 48-base-pair VNTR in DRD4 exon 3. The COMT met allele has been associated with reduced catechol-O-methyltransferase efficiency (Chen et al., 2004), likely increasing extrasynaptic DA accumulation, and with heightened DA receptor sensitivity among AUD individuals. The ANKK1 Taq1A A1 allele has been associated with dysregulated DA response among AUD individuals and is in high linkage disequilibrium with the DRD2 rs1076560 T allele, which has been associated with reduced striatal expression of the short (primarily presynaptic) isoform of the D₂ receptor, likely increasing extrasynaptic DA accumulation. Finally, the DRD4 VNTR long allele (i.e., the 48-base-pair VNTR allele) has been associated with reduced DRD4 mRNA expression, altered intracellular signaling after D₄ binding, and, among heavy drinkers, greater alcohol craving after consumption of a priming drink, greater alcohol cue-elicited striatal activation, and less cortical activation during response inhibition.

In some embodiments, the presently disclosed subject matter investigated whether DAT1/SLC6A3 VNTR genotype or a broader index of DA-related genetic variation comprising genotypes at the DAT1/SLC6A3 and DRD4 VNTRs and COMT rs4680 and DRD2 rs1076560 SNPs moderated APZ effects on reward-related phenotypes among non-treatment-seeking AUD individuals. Primary outcomes were alcohol cue-elicited VS activation and alcohol self-administration in a bar-lab setting. Although APZ has fewer adverse side effects than other atypical antipsychotics, it has been associated with some adverse effects (Mallikaarjun et al., 2004); thus, the severity of these effects was a secondary outcome. APZ, relative to placebo, was hypothesized to reduce all outcomes to a greater extent among DAT1/SLC6A3 9R carriers, relative to 10R homozygotes, and among individuals with a greater number of alleles associated with relatively higher basal DA tone.

Accordingly, in one aspect, disclosed herein are methods for treating a psychiatric, mental, and/or neurological disorder (such as, for example, AUD) or methods for detecting susceptibility to dopamine treatment further comprising assaying for polymorphisms in the genes encoding the DA-catabolizing enzyme catechol-O-methyltransferase (COMT) and the D₂ and D₄ receptors (DRD2 and DRD4).

### V. Nucleic Acids

There are a variety of molecules disclosed herein that are nucleic acid based, including for example the nucleic acids that encode, for example primers that hybridize to gene products derived from the DAT1/SLC6A3 gene, the COMT gene, the DRD2 gene, the DRD4 gene, and/or the ANKK1 gene. The disclosed nucleic acids are made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if, for example, an antisense molecule is introduced into a cell or cell environment through for example exogenous delivery, it is advantageous that the antisense molecule be made up of nucleotide analogs that reduce the degradation of the antisense molecule in the cellular environment.

V.A. Nucleotides and Related Molecules

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. A non-limiting example of a nucleotide would be 3′-AMP (3′-adenosine monophosphate) or 5′-GMP (5′-guanosine monophosphate). There are many varieties of these types of molecules available in the art and available herein.

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties. There are many varieties of these types of molecules available in the art and available herein.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid. There are many varieties of these types of molecules available in the art and available herein.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., 1989). There are many varieties of these types of molecules available in the art and available herein.

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH₂ or O) at the C6 position of purine nucleotides.

V.B. Primers and Probes

Disclosed are compositions including primers and probes, which are capable of interacting with the disclosed nucleic acids, such as the DAT1/SLC6A3 primers (SEQ ID NOs: 1 and 23 and SEQ ID NO: 2) and the DRD4 primers (SEQ ID NOs: 3 and 4) as disclosed herein. In certain embodiments the primers are used to support DNA amplification reactions. Typically, the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically, the disclosed primers hybridize with the disclosed nucleic acids or region of the nucleic acids or they hybridize with the complement of the nucleic acids or complement of a region of the nucleic acids.

In some embodiments, the size of the primers or probes for interaction with the nucleic acids can be any size that supports the desired enzymatic manipulation of the primer, such as DNA amplification or the simple hybridization of the probe or primer. A typical primer or probe would be in some embodiments at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

In some embodiments, a primer or probe can be less than or equal to 6, 7, 8, 9, 10, 11, 12 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

The primers for the DAT1/SLC6A3 gene typically are used to produce an amplified DNA product that contains a region of the DAT1/SLC6A3 gene or the complete gene. In general, typically the size of the product is such that the size can be accurately determined to in some embodiments within 3 nucleotides, in some embodiments within 2 nucleotides, and in some embodiments within 1 nucleotide.

In some embodiments, this product is at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

In some embodiments, the product is less than or equal to 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

### VI. Kits

In some embodiments, disclosed herein are kits that are drawn to reagents that can be used in practicing the methods disclosed herein. The kits can include any reagent or combination of reagent discussed herein or that would be understood to be required or beneficial in the practice of the disclosed methods. For example, the kits could include primers to perform the amplification reactions discussed in certain embodiments of the methods, as well as the buffers and enzymes required to use the primers as intended. For example, disclosed is a kit for treating a psychiatric, mental, and/or neurological disorder (such as, for example, AUD) or detecting susceptibility to dopamine treatment disclosed herein, comprising the primers set forth in SEQ ID NO: 1 and/or 23 and SEQ ID NO: 2 for the DAT1/SLC6A3 VNTR and SEQ ID NOs: 3 and 4 for the DRD4 VNTR. In some embodiments, the disclosed kits for treating a psychiatric, mental, and/or neurological disorder (such as, for example, AUD) can also comprise a dopamine modulator (such as, for example aripiprazole, brexipiprizole, and cariprazine).

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Methods and Materials for the Examples

The study was an eight-day sub-acute dosing human laboratory investigation (ClinicalTrials.gov identifier: NCT01292057). After randomization to study medication, subjects underwent an fMRI alcohol cue-reactivity task on day 7 of medication ingestion and a bar-lab paradigm on day 8. To maximize cue-reactivity and motivation to drink, they were instructed to abstain from alcohol on the evenings of days 6 and 7.

Subjects. Subjects were recruited via media advertisements, and were required to be ages 21-40 and to meet DSM-IV (Diagnostic and Statistical Manual of Mental Disorders, Revised 4^{th} Edition) diagnostic criteria for Alcohol Dependence, as assessed by the Structured Clinical Interview for DSM-IV. Exclusion criteria were: current DSM-IV Substance Dependence for any substance except nicotine; current use of illicit substances or psychotropic medications, as evidenced by urine drug screen and self-report; current DSM-IV Axis I diagnosis or suicidal/homicidal ideation; history of significant medical illness; and liver enzyme (ALT or AST) levels greater than three times normal. Female subjects could not be pregnant or nursing. At baseline, the Alcohol Dependence Scale, Obsessive Compulsive Drinking Scale (OCDS), and Timeline Follow-back were used to assess AUD severity, alcohol craving, and past-90-day drinking, respectively.

Ninety-nine subjects were randomized to medication, of whom two did not return after day 1. DNA was not collected from one subject. Of the remaining 96 subjects, two were discontinued from the study before completing the fMRI or bar-lab sessions (one had a positive breath alcohol concentration (BAC) on day 7 despite instructions to abstain, and one sustained a concussion that day), leaving a final sample of 94 for the bar-lab analyses (see Table 1). Of these individuals, six were not scanned due to equipment issues (n=2), claustrophobia (n=2), or alcohol withdrawal symptoms on day 7 (n=2), and seven were excluded due to motion during the scan, leaving a final sample of 81 for the imaging analyses.

TABLE 1
Demographic, Severity, and Drinking Data

| | DAT1/SLC6A3 | | DAT1/SLC6A3 | | | |
|---|---|---|---|---|---|---|
| | 9R carriers | | 10R homozygotes | | | |
| | Placebo | APZ | Placebo | APZ | P^{a} | P^{b} |
| N | 24 | 28 | 24 | 18 | — | — |
| Age | 27.9 | 25.4 | 26.1 | 28.4 | 0.69 | 0.18 |
| | (5.4) | (4.7) | (5.2) | (6.6) | | |
| Sex (% male) | 75.0 | 75.0 | 79.2 | 77.8 | 0.91 | 0.98 |
| Race | 75.0 | 82.1 | 83.3 | 83.3 | 0.67 | 0.87 |
| (% Caucasian) | | | | | | |
| Current Smoker | 58.3 | 35.7 | 33.3 | 55.6 | 0.82 | 0.19 |
| (%) | | | | | | |
| ADS Score | 11.3 | 13.2 | 12.0 | 12.6 | 0.29 | 0.72 |
| | (6.8) | (5.9) | (6.2) | (5.8) | | |
| BIS-11 Score | 63.8 | 67.2 | 66.7 | 64.1 | 0.80 | 0.78 |
| | (16.5) | (15.2) | (12.5) | (11.5) | | |
| OCDS Score | 16.4 | 18.0 | 16.3 | 19.2 | 0.22 | 0.63 |
| | (8.0) | (8.6) | (8.8) | (7.7) | | |
| Drinks per day^{c} | 6.2 | 8.6 | 7.2 | 7.8 | 0.02 | 0.05 |
| | (2.8) | (3.9) | (2.8) | (2.9) | | |
| Drinks per | 7.9 | 10.7 | 9.5 | 9.3 | 0.04 | 0.04 |
| drinking day^{c} | (3.1) | (3.4) | (3.6) | (3.4) | | |
| Heavy drinking | 72.3 | 94.4 | 80.1 | 79.5 | 0.01 | 0.01 |
| days %^{c} | (26.6) | (9.8) | (23.0) | (26.9) | | |

Abbreviations: APZ, aripiprazole; ADS, Alcohol Dependence Scale; BIS-11, Barratt Impulsiveness Scale; OCDS, Obsessive Compulsive Drinking Scale. Figures are means (standard deviations) unless otherwise indicated. Current smoking was defined as smoking ≥10 cigarettes per day. Statistics for differences between groups refer to the significance of the χ2 statistic for sex, race, and smoking and the t and F statistics for other variables.
P^{a} = Test for difference between medication groups
P^{b} = Test for difference between all four groups
^{c}In the 90 days prior to medication randomization

Genotyping. Genomic DNA was extracted from peripheral blood mononuclear cells (GENTRA® PURAGENE® Blood Kit brand; Qiagen Inc., Valencia, Calif., United States of America) and amplified by polymerase-chain-reaction (PCR). VNTR genotypes were determined using custom primers 5′-TGTGGTGTAGGGAACGGCCTGAG-3′ (SEQ ID NO: 1) or 5′-TGCGGTGTAGGGAACGGCCTGAG-3 (SEQ ID NO: 23) in conjunction with 5′-CTTCCTGGAGGTCACGGCTCAAGG-3′ (SEQ ID NO: 2) for DAT1/SLC6A3 and 5′-AGGACCCTCATGGCCTTG-3′ (SEQ ID NO: 3) and 5′-GCGACTACGTGGTCTACTCG-3′ (SEQ ID NO: 4) for DRD4. (Thermo Fisher Scientific, Waltham, Mass., United States of America). Amplified samples were electrophoresed on 2.0% agarose gels and visualized with ethidium bromide under ultraviolet light, and genotypes were scored by two raters independently. For DAT1/SLC6A3, two subjects carried alleles other than 9R or 10R: one had one 8R allele, and one had two 3R alleles. Since alleles with fewer than 9 repeats have also been associated with reduced DAT expression relative to the 10R allele (Fuke et al., 2001), these alleles were categorized as 9R for analytic purposes. For DRD4, using the classification system most consistent in the addiction literature, alleles were scored as “long” (≥7 repeats) or “short” (<7 repeats). DRD4 genotype could not be determined for one subject. SNP genotypes were determined with a STEPONE™ brand Real-Time PCR System and TAQMAN™ brand 5′ nuclease assays (Thermo Fisher Scientific, Waltham, Mass., United States of America), using allele-specific probes (Catalog #4351376 and #4362691) and three known controls for each genotype. Genotypes for all polymorphisms (Table 2) were in Hardy-Weinberg equilibrium and consistent with expected population allele frequencies.

TABLE 2
Genotype Frequencies and Scoring* for Each Polymorphism

| | | | | DA-related |
|---|---|---|---|---|
| Polymorphism | Genotypes | N | Frequency | composite score |
| DAT1/SLC6A3 VNTR | 9/9 | 16 | 0.17 | High |
| | 9/10 | 36 | 0.38 | Intermediate |
| | 10/10 | 42 | 0.45 | Low |
| COMT rs4680 | Met/Met | 25 | 0.27 | High |
| | Met/Val | 41 | 0.44 | Intermediate |
| | Val/Val | 28 | 0.3 | Low |
| DRD2 rs1076560 | T/T | 1 | 0.01 | High |
| | T/G | 27 | 0.29 | Intermediate |
| | G/G | 66 | 0.70 | Low |
| DRD4 VNTR | Long/Long | 3 | 0.03 | High |
| | Long/Short | 29 | 0.31 | Intermediate |
| | Short/Short | 61 | 0.65 | Low |

*For the DA-related genetic composite measure, “high” genotypes (2 alleles associated with higher DA tone) were scored as 2, “intermediate” genotypes as 1, and “low” genotypes as 0. The total possible score for the composite ranged from 0 (no higher-DA alleles) to 8 (all higher-DA alleles).

Randomization and medication. Subjects were urn randomized to receive APZ (day 1: 5 mg; days 2-3: 10 mg; days 4-8: 15 mg) or placebo for eight days, and were observed to ingest the first and last medication doses. Subjects and investigators were blind to both genotype and medication assignment. Thus, subjects were stratified by their baseline Barratt Impulsiveness Scale (BIS-11) score (Patton et al., 1995) into groups with BIS-11 scores greater vs. less than the median score of 68 and randomization was conducted separately within each stratum. Urn variables balanced across medication groups within each stratum were sex and smoking status. Study medications were identically over-encapsulated with 100 mg riboflavin for adherence measurement (which was high and did not significantly differ between medication groups) and distributed in labeled blister packs.

Neuroimaging. On day 7, subjects were breathalyzed, assessed for alcohol withdrawal, and re-administered the OCDS. As noted above, one subject with a BAC>0 and two subjects with Clinical Institute Withdrawal Assessment for Alcohol-Revised scores >4 were excluded from scanning. After acquisition of a high-resolution anatomical image, subjects were given a sip (10 ml) of their preferred 80-proof liquor mixed with fruit juice and administered a 12-m-long task during which they passively viewed pseudo-randomly interspersed blocks of alcoholic beverage images (ALC; equally distributed between beer, wine, and liquor), nonalcoholic beverage images (BEV), blurred versions of these images that served as visual controls, and a fixation cross. Each 24-s-long block comprised only one image type and was followed by a 6-s period during which subjects were instructed to rate their urge for alcohol. Images were selected from a normative set, supplemented with images from advertisements, and matched for intensity, color, and complexity. This task consistently elicits robust cue-elicited VS activation among non-treatment-seeking AUD individuals.

Functional images were acquired with a gradient echo, echo-planar imaging sequence implemented on a 3T TIM Trio scanner (Siemens, Erlangen, Germany). Acquisition parameters were: repetition/echo times=2200/35 ms; 328 volumes; flip angle=90°; field of view=192 mm; matrix=64×64; voxel size=3.0×3.0 mm; 36 contiguous 3.0-mm-thick transverse slices. Using FEAT (fMRI Expert Analysis Tool) v. 6.00, part of FSL (FMRIB Software Library, University of Oxford, United Kingdom), functional images were realigned to the middle volume, spatially smoothed (8-mm full width at half maximum kernel), resampled to 2-mm isotropic voxels, and registered, first to the subject's high-resolution anatomical image and subsequently to the Montreal Neurological Institute (MNI; Montréal, Canada) 152-subject-average template. Based on previous data indicating an interaction between DAT1/SLC6A3 genotype and naltrexone on alcohol cue-elicited VS activation, the right VS was defined a priori as a 6-mm-radius sphere centered at the point [12 15 −6] in MNI space. For each subject, this sphere was reverse-registered from the MNI-152 image to the subject's anatomical image, and the average percentage change of the blood-oxygen-level-dependent signal between ALC and BEV blocks (i.e., ALC vs. BEV percent signal change) was extracted.

Bar-lab paradigm. On day 8, subjects were observed to ingest the last medication dose at 11:30 AM. Thirty minutes later, they were provided a standard caloric lunch, adjusted for gender and weight. At 2:00 PM, subjects were administered a priming drink of their preferred 80-proof liquor in a 1:3 ratio with juice, adjusted for gender, age, and weight to produce a targeted BAC of 30 mg %, and instructed to consume it within five minutes. Forty minutes later, subjects were presented a tray of four drinks, each with a targeted BAC of 15 mg %, and told they could consume as many as they desired over the next hour. After an hour, this tray was removed and another tray of four drinks was made available for consumption over a second hour. To create a decisional balance between drinking and abstaining, subjects were given a “bar credit” of $16 with which to “purchase” drinks, at the cost of $2/drink. After the procedure, subjects were given dinner and remained until 10:00 PM. A BAC measurement below 20 mg % was required before departure, and a friend or taxi drove subjects home.

Adverse effects. A physical symptom checklist was used to assess the presence and severity (self-rated as none, mild, moderate, or severe) of 21 possible adverse effects at baseline and on day 8 immediately before the bar-lab paradigm.

Statistical analysis. The general linear model (GLM; SPSS 23, IBM, Armonk, N.Y., United States of America) was used to test the interaction between medication and DAT1/SLC6A3 genotype on the primary outcomes (VS ALC vs. BEV activation and the number of drinks consumed in the bar lab). A model that included between-subjects factors for medication, DAT1/SLC6A3 genotype, and their interaction was tested for each outcome. Analyses first compared 9R carriers to 10R homozygotes, and subsequently examined the additive effect of the 9R allele. By chance, baseline drinking significantly differed between medication groups (Table 1); accordingly, baseline drinks per day was co-varied in all models. Significant interactions were followed up with simple effects contrasts. VS activation was also analyzed for correlation (Pearson's r) with day 7 OCDS scores and bar-lab drinking.

The GLM was also used to test the interaction between medication and a DA-related genetic composite on the primary outcomes. The DAT1/SLC6A3 9R, COMT met, DRD2 T, and DRD4 long alleles were categorized as higher-DA alleles, and the total number of alleles each subject carried was calculated (Table 2). Few subjects carried more than four higher-DA alleles (four subjects had five alleles and one had seven), so these subjects were combined with those who carried four, yielding five groups of subjects with zero (n=7), one (n=15), two (n=31), three (n=22), or four or more (n=18) alleles. A model that included between-subjects factors for medication, the additive effect of the number of higher-DA alleles, and their interaction was tested for each outcome.

For the secondary outcome (adverse effect severity), the GLM was first used to evaluate the main effect of medication on the presence/absence of each effect. Insomnia, daytime sleepiness, irritability, trouble concentrating, nausea/vomiting, dizziness, fatigue, blurry vision, and difficulty reaching orgasm were significantly more frequent (p<0.05) in the APZ group. To account for the influence of these effects on the pharmacogenetic interactions evaluated above, the presence/absence of each was evaluated as a covariate in all models. To evaluate whether DAT1/SLC6A3 genotype or DA composite score affected adverse effect severity, the generalized linear model, which can accommodate an ordinal logistic dependent variable, was used to test interactions between medication and DAT1/SLC6A3 genotype and between medication and the additive effect of the number of higher-DA alleles on the severity of each effect that significantly differed between medication groups.

### EXAMPLE 1

### Cue-Elicited VS Activation

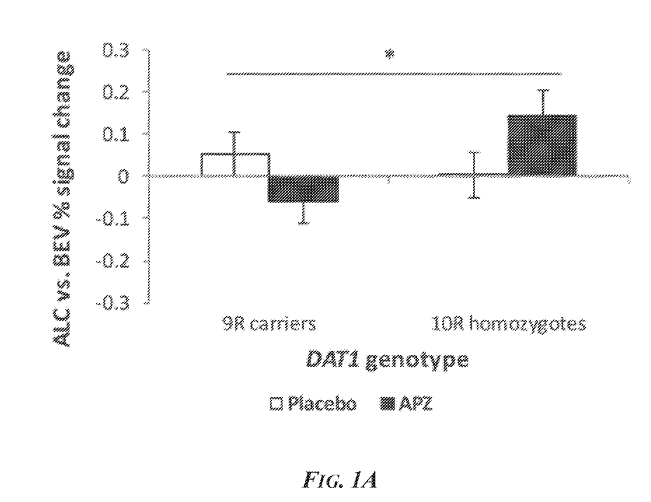

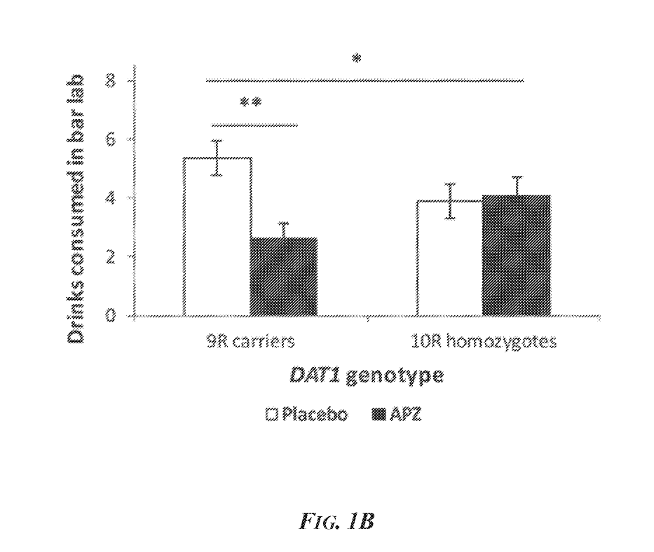

The interaction between medication and DAT1/SLC6A3 genotype was significant (F(1, 76)=4.98, p=0.029, partial η2=0.061). Relative to placebo, APZ reduced VS activation among 9R carriers, but increased it among 10R homozygotes (FIG. 1A). The simple effect of medication approached significance among 10R homozygotes: (F(1, 76)=2.86, p=0.095). In additive analyses, the interaction between medication and the additive effect of the 9R allele was significant (F(1, 76)=5.13, p=0.026, partial η2=0.067), such that APZ, relative to placebo, reduced VS activation more among subjects with a greater number of 9R alleles. VS activation was significantly positively associated with day 7 OCDS score (r(81)=0.26, p=0.018), but not bar-lab drinking.

### EXAMPLE 2

### Bar-Lab Drinking

The main effect of medication (F(1, 89)=4.63, p=0.034, partial ρ2=0.049) and the interaction between medication and DAT1/SLC6A3 genotype (F(1, 89)=6.11, p=0.015, partial η2=0.064) were significant, such that APZ, relative to placebo, reduced drinking among 9R carriers, but not 10R homozygotes (FIG. 1B). The simple effect of medication was significant only among 9R carriers (F(1, 89)=11.54, p=0.001, partial η2=0.12). In additive analyses, the interaction between medication and the additive effect of the 9R allele was significant (F(1, 89)=4.73, p=0.032, partial η2=0.068), such that APZ, relative to placebo, reduced drinking more among subjects with a greater number of 9R alleles. A model that included BIS-11 self-control scale score (median split), DAT1/SLC6A3 genotype, and medication was tested; the interactions between genotype and medication (F(1, 85)=4.87, p=0.030, partial η2=0.054) and between self-control and medication F(1, 85)=4.86, p=0.030, partial η2=0.054) were each significant, but the three-way interaction between these factors was not, indicating that the DAT1/SLC6A3 and self-control effects were independent from each other. Self-control also did not significantly moderate the medication by DAT1/SLC6A3 interaction on VS activation.

### EXAMPLE 3

### DA-Related Genetic Composite Effects

The interactions between medication and the additive effect of higher-DA alleles was also determined. The results are presented in Table 3.

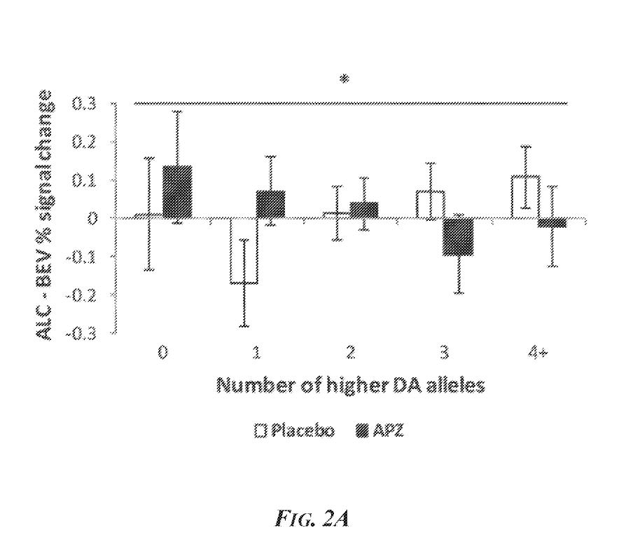

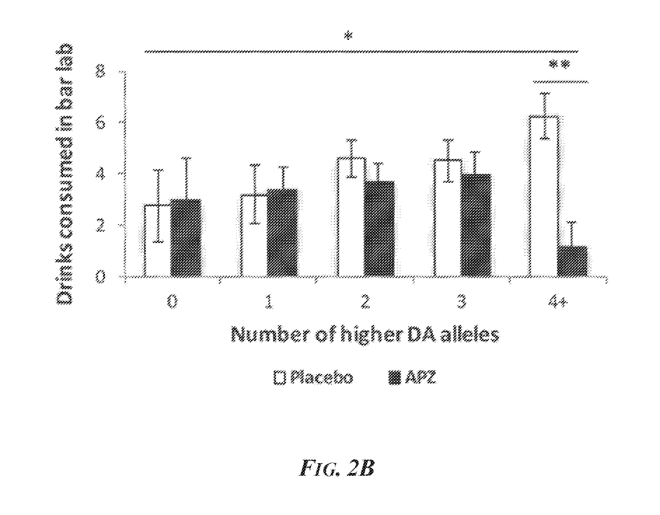

The interactions between medication and the additive effect of higher-DA alleles was significant for both VS activation (F(1, 76)=4.12, p=0.046, partial ηₚ²=0.051; see FIG. 2A) and bar-lab drinking (F(1, 88)=6.50, p=0.013, partial ηₚ²=0.069; see FIG. 2A), such that APZ reduced these outcomes more among subjects who carried a greater number of higher-DA alleles. The simple effect of medication on bar-lab drinking was significant only among subjects who carried four or more higher-DA alleles (F(1, 82)=14.60, p=0.0005, partial ηₚ²=0.15).

TABLE 3
P Values and Effect Sizes for Interactions Between Medication and
Permutations of DAT1 VNTR and Other DA-related Polymorphisms

| | VS Activation | | Bar Lab Drinks | |
|---|---|---|---|---|
| | (n = 81) | | (n = 94) | |
| Genotype | P | □ₚ² | p | □ₚ² |
| DAT1 | 0.026 | 0.063 | 0.032 | 0.050 |
| DAT1 + COMT | 0.19 | 0.023 | 0.007 | 0.079 |
| DAT1 + DRD2 | 0.021 | 0.068 | 0.006 | 0.081 |
| DAT1 + DRD4 | 0.014 | 0.077 | 0.045 | 0.045 |
| DAT1 + COMT + DRD2 | 0.16 | 0.026 | 0.005 | 0.100 |
| DAT1 + COMT + DRD4 | 0.087 | 0.038 | 0.011 | 0.071 |
| DAT1 + DRD2 + DRD4 | 0.01 | 0.084 | 0.011 | 0.072 |
| DAT1 + COMT + DRD2 + DRD4 | 0.046 | 0.051 | 0.013 | 0.069 |

Statistics are for the interaction between medication (aripiprazole vs. placebo) and each permutation of genotypes (additive effect of number of DAT1 9R, COMT met, DRD2 T, and/or DRD4 long alleles) in a general linear model that also included baseline drinks per day and the main effects of medication and the genotype permutation. P values for significant interactions (p<0.05) are bolded.

### EXAMPLE 4

### Adverse Effects

All interactions described above remained significant when any or all of the effects that significantly differed between medication groups were covaried. The results are summarized in Table 4.

The interaction between medication and DAT1/SLC6A3 genotype on insomnia severity was significant (Wald _{χ}2(1, N=94)=7.77, p=0.005), such that 10R homozygotes who received APZ, relative to placebo, had more severe insomnia, but 9R carriers did not (4). The interaction between medication and DAT1/SLC6A3 genotype on irritability approached significance (Wald _{χ}2(1, N=94)=2.92, p=0.088), in the same direction as the interaction for insomnia. DAT1/SLC6A3 genotype did not significantly moderate the severity of any other effects that significantly differed between medication groups. There was also a significant interaction between medication and the additive effect of higher-DA alleles on insomnia severity (Wald _{χ}2(1, N=93)=5.01, p=0.025), but not other adverse effects, such that, among subjects who received APZ, insomnia was less severe among subjects with a greater number of higher-DA alleles.

TABLE 4
Number of Subjects with Varying Severity of Insomnia
by DAT1/SLC6A3 Genotype and Medication Group

| | |
|---|---|
| 9R carriers | 10R homozygotes |

| Severity | Placebo | APZ | Placebo | APZ |
|---|---|---|---|---|
| None | 12 | 11 | 16 | 2 |
| Mild | 7 | 11 | 4 | 8 |
| Moderate | 5 | 6 | 3 | 4 |
| Severe | 0 | 0 | 1 | 4 |

*p < 0.05 for interaction between DAT1/SLC6A3 genotype and medication group on insomnia severity (generalized linear model; ordinal outcome).

### EXAMPLE 5

### Race Effects

Since the frequency of the DAT1/SLC6A3 9R and other higher-DA alleles varies by race, all models were evaluated among only subjects with self-reported Caucasian ancestry (n=76 for those with bar-lab data and n=66 for those with usable imaging data). All effects that were statistically significant in the larger sample remained significant in this subsample except for the interaction between medication and the DA-related composite on VS activation, which was reduced to trend-level significance (p=0.066).

### Discussion of the Examples

Collectively, these data indicate a novel pharmacogenetic interaction between DA-related genetic variation and APZ response in AUD. This interaction was present for both the DAT1/SLC6A3 VNTR and a composite measure that aggregated genotypes at this VNTR and three other putatively functional polymorphisms in DA-related genes. In each case, individuals who carried a greater number of alleles associated with higher basal DA tone displayed better APZ effects. Thus, APZ may be beneficial among individuals genetically predisposed to enhanced DA effects, but ineffective among others.

DAT1/SLC6A3 9R carriers treated with APZ, compared to placebo, displayed less cue-elicited VS activation and alcohol self-administration, and reported less severe APZ-related insomnia, than 10R homozygotes. Given the 9R allele's association with lower DAT expression and enhanced reward-related VS activation, these individuals might have been predisposed to greater striatal extrasynaptic DA accumulation or prolonged effects after exposure to alcohol cues or the priming drink administered in the bar lab. APZ's DA partial agonist effect thus displaced endogenous DA among these individuals, reducing DA-mediated cue reactivity or alcohol reward. Although the interaction between medication and DAT1/SLC6A3 genotype was significant for both cue-elicited VS activation and bar-lab drinking, these outcomes were not significantly correlated, indicating potentially dissociable effects. However, since bar-lab safety constraints limited the number of drinks available for self-administration, the upper range of this variable might have been artificially restricted, obviating correlation with (unrestricted) VS activation.

Beyond its interaction with DAT1/SLC6A3 genotype, APZ, as hypothesized, also more effectively reduced VS activation and self-administration, and caused less severe insomnia, among individuals who carried a greater number of alleles associated with higher DA tone. Its effect size on bar-lab drinking was greater among individuals with four or more higher-DA alleles (partial η2=0.15) than among DAT1/SLC6A3 9R carriers (partial η2=0.12), indicating that the genetic composite accounted for a greater proportion of the variance in APZ effects than DAT1/SLC6A3 genotype alone. An interaction in the opposite direction was reported between a similar DA-related genetic composite and the effects of the D₂/D₃ full agonist ropinirole on impulsive decision-making, such that ropinirole, relative to placebo, increased impulsive decisions among healthy adults who carried more higher-DA alleles. These findings indicate that individuals genetically predisposed to high DA tone can be hypersensitive to reward, and that partial, but not full, DA agonists can be beneficial in reducing this hypersensitivity among these individuals, perhaps by normalizing DA tone.

This study had several strengths, including low attrition and the use of well-validated alcohol cue-reactivity and self-administration paradigms. However, several factors limit interpretation. First, subjects were non-treatment-seeking individuals who were compensated for participation; it is unclear whether these pharmacogenetic findings extend to treatment-seeking individuals, such as those examined in the multi-site APZ trial. Second, DA-related genetic variation was an exploratory study aim, and subjects were randomized to medication by their level of trait impulsivity, rather than DAT1/SLC6A3 genotype or their DA-related composite score. However, impulsivity was well-balanced between groups and did not significantly moderate the interactions between medication and DAT1/SLC6A3 genotype or the DA-related composite. Finally, several caveats regarding the composite should be noted. This measure assumed additive, rather than interactive, effects of its constituent polymorphisms, and combined polymorphisms associated with changes in both striatal and cortical DA. An interactive approach would have allowed evaluation of epistatic effects and of potential differences in APZ effects as a function of striatal vs. cortical DA tone. However, the low minor allele frequencies for the DRD2 and DRD4 polymorphisms precluded this strategy.

In conclusion, this study indicated that, among non-treatment-seeking young adults with AUD, polymorphisms in DAT1/SLC6A3 and other DA-related genes moderated the effects of APZ on alcohol cue-elicited striatal activation and alcohol self-administration. Further exploration of the effects of DA-related genetic variation on APZ efficacy in AUD is warranted.

### REFERENCES

All references listed below, as well as all references cited in the instant disclosure, including but not limited to all patents, patent applications and publications thereof, scientific journal articles, and database entries (e.g., GENBANK® database entries and all annotations available therein) are incorporated herein by reference in their entireties to the extent that they supplement, explain, provide a background for, or teach methodology, techniques, and/or compositions employed herein.

Aarts et al. (2010) Striatal dopamine mediates the interface between motivational and cognitive control in humans: evidence from genetic imaging. Neuropsychopharmacology. 35:1943-1951.
Anton et al. (1996) The obsessive compulsive drinking scale: A new method of assessing outcome in alcoholism treatment studies. Arch Gen Psychiatry. 53:225-231.
Anton et al. (2008) A randomized, multicenter, double-blind, placebo-controlled study of the efficacy and safety of aripiprazole for the treatment of alcohol dependence. J Clin Psychopharmacol. 28:5-12.
Anton et al. (2012) Naltrexone modification of drinking effects in a subacute treatment and bar-lab paradigm: influence of OPRM1 and dopamine transporter (SLC6A3) genes. Alcohol Clin Exp Res. 36:2000-2007.
Anton et al. (2017) Ariprazole suppression of drinking in a clinical laboratory paradigm: Influence of impulsivity and self-control. Alcohol Clin Exp Res. 41:1370-1380.
Asghari et al. (1995) Modulation of intracellular cyclic AMP levels by different human dopamine D₄ receptor variants. J Neurochem. 65:1157-1165.
Blasi et al. (2015) Variation in Dopamine D₂ and Serotonin 5-HT_{2A} Receptor Genes is Associated with Working Memory Processing and Response to Treatment with Antipsychotics. Neuropsychopharmacology. 40:1600-1608.
Boileau et al. (2003) Alcohol promotes dopamine release in the human nucleus accumbens. Synapse (New York, N.Y.). 49:226-231.
Brody et al. (2006) Gene variants of brain dopamine pathways and smoking-induced dopamine release in the ventral caudate/nucleus accumbens. Arch Gen Psychiatry. 63:808-816.
Chang et al. (1996) The world-wide distribution of allele frequencies at the human dopamine D₄ receptor locus. Hum Genet. 98:91-101.
Chen et al. (2004) Functional analysis of genetic variation in catechol-O-methyltransferase (COMT) effects on mRNA, protein, and enzyme activity in postmortem human brain. Am J Hum Genet. 75:807-821.
Clarke et al. (2014) The dopamine receptor D₂ (DRD2) SNP rs1076560 is associated with opioid addiction. Ann Hum Genet. 78:33-39.

### Diagnostic and Statistical Manual of Mental Disorders, 4th Edition, Text Revision (DSM-IV-TR). American Psychiatric Association Publishing, Washington, D.C., United States of America.

Dreher et al. (2009) Variation in dopamine genes influences responsivity of the human reward system. Proc Natl Acad Sci U S A. 106:617-622.
European Patent Publication No. EP 0 070 685 B1.
Faraone et al. (2014) Functional effects of dopamine transporter gene genotypes on in vivo dopamine transporter functioning: a meta-analysis. Mol Psychiatry. 19:880-889.
Filbey et al. (2008) Differential neural response to alcohol priming and alcohol taste cues is associated with DRD4 VNTR and OPRM1 genotypes. Alcohol Clin Exp Res. 32:1113-1123.
Filbey et al. (2011) Dopaminergic genes modulate response inhibition in alcohol abusing adults. Addict Biol. 17:1046-1056.
First et al. (2002) Structured Clinical Interview for DSM-IV-TR Axis I Disorders, Research Version, Non-patient Edition. New York: Biometrics Research, New York State Psychiatric Institute, New York, N.Y., United States of America.
Forbes et al. (2009) Genetic variation in components of dopamine neurotransmission impacts ventral striatal reactivity associated with impulsivity. Mol Psychiatry. 14:60-70.
Franklin et al. (2009) DAT genotype modulates brain and behavioral responses elicited by cigarette cues. Neuropsychopharmacology. 34:717-728.
Franklin et al. (2011) Dopamine transporter genotype modulation of neural responses to smoking cues: confirmation in a new cohort. Addict Biol. 16:308-322.
Fuke et al. (2001) The VNTR polymorphism of the human dopamine transporter (DAT1) gene affects gene expression. Pharmacogenom J. 1:152-156.
Gonzales & Weiss (1998) Suppression of ethanol-reinforced behavior by naltrexone is associated with attenuation of the ethanol-induced increase in dialysate dopamine levels in the nucleus accumbens. J Neurosci. 18:10663-10671.
Heinz et al. (2000) Genotype influences in vivo dopamine transporter availability in human striatum. Neuropsychopharmacology. 22:133-139.
Hutchison et al. (2002) The DRD4 VNTR polymorphism moderates craving after alcohol consumption. Health Psychol. 21:139-146.
Hutchison et al. (2003) Olanzapine reduces craving for alcohol: a DRD4 VNTR polymorphism by pharmacotherapy interaction. Neuropsychopharmacology. 28:1882-1888.
Ito et al. (2012) Effects of dopamine D₂ receptor partial agonist antipsychotic aripiprazole on dopamine synthesis in human brain measured by PET with L-[beta-11C]DOPA. PLoS One. 7:e46488.
Kang et al. (1999) Global variation of a 40-bp VNTR in the 3′-untranslated region of the dopamine transporter gene (SLC6A3). Biol Psychiatry. 46:151-160.
Kasparbauer et al. (2015) Methylphenidate effects on brain activity as a function of SLC6A3 genotype and striatal dopamine transporter availability. Neuropsychopharmacology. 40:736-745.
Khanna et al. (2014) Aripiprazole versus other atypical antipsychotics for schizophrenia. Cochrane Database Syst Rev. 2:CD006569.
Kooij et al. (2008) Response to methylphenidate in adults with ADHD is associated with a polymorphism in SLC6A3 (DAT1). Am J Med Genet B Neuropsychiatr Genet. 147B:201-208.
Kranzler et al. (2008) Effects of aripiprazole on subjective and physiological responses to alcohol. Alcohol Clin Exp Res. 32:573-579.
Letsinger et al. (1989) Cholesteryl-conjugated oligonucleotides: synthesis, properties, and activity as inhibitors of replication of human immunodeficiency virus in cell culture. Proc Natl Acad Sci U S A 86:6553-6556.
MacDonald et al. (2016) Dopamine Gene Profiling to Predict Impulse Control and Effects of Dopamine Agonist Ropinirole. J Cogn Neurosci. 28:909-919.
Mallikaarjun et al. (2004). Pharmacokinetics, tolerability, and safety of aripiprazole following multiple oral dosing in normal healthy volunteers. J Clin Pharmacol 44:179-187.
Martinez et al. (2005) Alcohol dependence is associated with blunted dopamine transmission in the ventral striatum. Biol Psychiatry. 58:779-786.
McGeary (2009) The DRD4 exon 3 VNTR polymorphism and addiction-related phenotypes: a review. Pharmacol Biochem Behay. 93:222-229.
Milos (2010) Helicos single molecule sequencing: unique capabilities and importance for molecular diagnostics. Genome Biol. 11(Suppl 1):I14.
Myrick et al. (2010) The effect of aripiprazole on cue-induced brain activation and drinking parameters in alcoholics. J Clin Psychopharmacol. 30:365-372.
Nikolova et al. (2011) Multilocus genetic profile for dopamine signaling predicts ventral striatum reactivity. Neuropsychopharmacology. 36:1940-1947.
Oberlin et al. (2013) Beer flavor provokes striatal dopamine release in male drinkers: mediation by family history of alcoholism. Neuropsychopharmacology. 38:1617-1624.
Palmatier et al. (1999) Global variation in the frequencies of functionally different catechol-O-methyltransferase alleles. Biol Psychiatry. 46:557-567.
Patton et al. (1995) Factor structure of the Barratt impulsiveness scale. J Clin Psychol. 51:768-774.
Quake et al. (2003) Sequence information can be obtained from single DNA molecules. Proc Natl Acad Sci U S A. 100:3960-3964.
Schacht (2016) COMT val158met moderation of dopaminergic drug effects on cognitive function: a critical review. Pharmacogenomics J. 16:430-438.
Schacht et al. (2011) Stability of fMRI striatal response to alcohol cues: a hierarchical linear modeling approach. Neurolmage. 56:61-68.
Schacht et al. (2013) Interacting effects of naltrexone and OPRM1 and DAT1 variation on the neural response to alcohol cues. Neuropsychopharmacology. 38:414-422.
Schellekens et al. (2012) Reduced dopamine receptor sensitivity as an intermediate phenotype in alcohol dependence and the role of the COMT Va1158Met and DRD2 Taq1A genotypes. Arch Gen Psychiatry. 69:339-348.
Shapiro et al. (2003) Aripiprazole, a novel atypical antipsychotic drug with a unique and robust pharmacology. Neuropsychopharmacology. 28:1400-1411.
Simpson et al. (2010) The DRD4 receptor Exon 3 VNTR and 5′ SNP variants and mRNA expression in human post-mortem brain tissue. Am J Med Genet B Neuropsychiatr Genet. 153B:1228-1233.
Skinner & Allen (1982) Alcohol dependence syndrome: measurement and validation. J Abnorm Psychol. 91:199-209.
Smith et al. (2004) Advances in functional and structural MR image analysis and implementation as FSL. Neurolmage. 23 Suppl 1:S208-219.
Sobell & Sobell (1992) Timeline follow-back: A technique for assessing self-reported alcohol consumption. in: Allen & Litten (eds). Measuring Alcohol Consumption: Psychosocial and Biochemical Methods. Humana Press, Totowa, N.J., United States of America. pages 41-72.
Stice et al. (2012) Multilocus genetic composite reflecting dopamine signaling capacity predicts reward circuitry responsivity. J Neurosci. 32:10093-10100.
Stout et al. (1994) Ensuring balanced distribution of prognostic factors in treatment outcome research. J Stud Alcohol Suppl. 12:70-75.
Stritzke et al. (2004) Assessment of substance cue reactivity: advances in reliability, specificity, and validity. Psychol Addict Behav. 18:148-159.
Sullivan et al. (1989) Assessment of alcohol withdrawal: the revised clinical institute withdrawal assessment for alcohol scale (CIWA-Ar). Br J Addict. 84:1353-1357.
Tyagi & Kramer (1996) Molecular beacons: probes that fluoresce upon hybridization. Nature Biotechnol. 14:303-308.

### U.S. Patent Application Publication No. 2009/0208957; 2010/0144836.

U.S. Pat. Nos. 4,683,195; 4,683,202; 4,965,188; 5,563,037; 6,013,445; 6,350,580; 7,211,414; 9,982,296.
van Holstein et al. (2011) Human cognitive flexibility depends on dopamine D₂ receptor signaling. Psychopharmacology (Berlin). 218:567-578.
Vergne & Anton (2010) Aripiprazole: a drug with a novel mechanism of action and possible efficacy for alcohol dependence. CNS & neurological disorders drug targets. 9:50-54.
Volkow et al. (2007) Profound decreases in dopamine release in striatum in detoxified alcoholics: possible orbitofrontal involvement. J Neurosci. 27:12700-12706.
Voronin et al. (2008) Aripiprazole effects on alcohol consumption and subjective reports in a clinical laboratory paradigm—possible influence of self-control. Alcohol Clin Exp Res. 32:1954-1961.
Watson (1989) Total body water and blood alcohol levels: Updating the fundamentals. In: Crow K E, Batt R D, editors. Human Metabolism of Alcohol (Vol 1) Pharmacokinetics, medicolegal aspects, and general interest. Boca Raton: CRC Press.

Wong et al. (2014) A phase 2, placebo-controlled study of the opioid receptor antagonist LY2196044 for the treatment of alcohol dependence. Alcohol Clin Exp Res. 38:511-520.

Yacubian et al. (2007) Gene-gene interaction associated with neural reward sensitivity. Proc Natl Acad Sci U S A. 104:8125-8130.
Yoder et al. (2016) Differences in IV alcohol-induced dopamine release in the ventral striatum of social drinkers and nontreatment-seeking alcoholics. Drug Alcohol Depend. 160:163-169.
Yokoi et al. (2002) Dopamine D₂ and D₃ receptor occupancy in normal humans treated with the antipsychotic drug aripiprazole (OPC 14597) a study using positron emission tomography and [11C] raclopride. Neuropsychopharmacology. 27:248-259.
Zhang et al. (2007) Polymorphisms in human dopamine D₂ receptor gene affect gene expression, splicing, and neuronal activity during working memory. Proc Natl Acad Sci U S A. 104:20552-20557.

It will be understood that various details of the presently disclosed subject matter can be changed without departing from the scope of the presently disclosed subject matter. Furthermore, the foregoing description is for the purpose of illustration only, and not for the purpose of limitation.

What is claimed is:

1. A method for treating a subject with a disorder associated with dopamine receptor activity, the method comprising:
(a) performing or having performed one or more genotyping assays on a nucleic acid sample isolated from the subject to determine the subject's genotype with respect to a variable number tandem repeats (VNTR) polymorphism in a dopamine transporter DAT1/SLC6A3 gene, an rs4680 polymorphism in a DA-catabolizing enzyme catechol-O-methyltransferase (COMT) gene, an rs1076560 polymorphism in a D₂ receptor (DRD2) gene, a 48-base-pair VNTR polymorphism in a D₄ receptor (DRD4) gene, and/or an rs1800497 polymorphism in an ankyrin repeat and kinase domain containing 1 (ANKK1) gene; and
(b) administering a dopamine partial agonist to the subject if the one or more genotyping assays indicates that subject's genotype includes:
(i) at least one allele for 9 tandem repeats of the DAT1/SLC6A3 VNTR; or
(ii) four or more of: (1) a 9 tandem repeat allele of the DAT1/SLC6A3 VNTR; (2) a COMT A allele of the rs4680 polymorphism; (3) a 48-base-pair VNTR in DRD4 exon 3 allele; and (4) a DRD2 T allele of the rs1076560 polymorphism, an ANKK1 TaqA1 A allele of the rs1800497 polymorphism, or both.

2. The method of claim 1, wherein the disorder associated with dopamine receptor activity is an alcohol use disorder (AUD).

3. The method of claim 1, wherein the one or more genotyping assays are performed prior to administering the dopamine partial agonist.

4. The method of claim 1, wherein the one or more genotyping assays are performed after initiating a dopamine partial agonist therapy, and further wherein if the subject is homozygous for a DAT1/SLC6A3 VNTR 10 tandem repeat allele, the dopamine partial agonist therapy is discontinued.

5. The method of claim 1, wherein the dopamine partial agonist is selected from the group consisting of aripiprazole, brexipiprizole, and cariprazine.

6. The method of claim 1, wherein at least one of the one or more genotyping assays comprises a nucleic acid amplification process followed by sequencing or gel electrophoresis of a resulting nucleic acid amplification product.

7. The method of claim 1, wherein the one or more genotyping assays determine the subject's genotype with respect to the VNTR polymorphism in the dopamine transporter DAT1/SLC6A3 gene, the rs1076560 polymorphism in the DRD2 gene, and the 48-base-pair VNTR polymorphism in the DRD4 gene.

8. The method of claim 7, wherein the one or more genotyping assays further comprise a genotyping assay that determines the subject's genome with respect to the rs4680 polymorphism in the COMT gene.

9. A method for detecting a susceptibility to a dopamine partial agonist therapy in a subject suffering from or at risk for developing a disorder associated with dopamine receptor activity, the method comprising:
(a) obtaining a biological sample from the subject; and
(b) performing or having performed one or more genotyping assays on a nucleic acid sample isolated from the subject to determine the subject's genotype with respect to a variable number tandem repeats (VNTR) polymorphism in a dopamine transporter DAT1/SLC6A3 gene, an rs4680 polymorphism in a DA-catabolizing enzyme catechol-O-methyltransferase (COMT) gene, an rs1076560 polymorphism in a D₂ receptor (DRD2) gene, a 48-base-pair VNTR polymorphism in a D₄ receptor (DRD4) gene, and/or an rs1800497 polymorphism in an ankyrin repeat and kinase domain containing 1 (ANKK1) gene,
wherein detection of at least one allele for 9 tandem repeats of the DAT1/SLC6A3 VNTR or four or more of (1) a 9 tandem repeat allele of the DAT1/SLC6A3 VNTR; (2) a COMT A allele of the rs4680 polymorphism; (3) a 48-base-pair VNTR in DRD4 exon 3 allele; and (4) a DRD2 T allele of the rs1076560 polymorphism, an ANKK1 TaqA1 A allele of the rs1800497 polymorphism, or both, indicates that the subject is susceptible to a dopamine partial agonist therapy.

10. The method of claim 9, wherein the dopamine partial agonist is selected from the group consisting of aripiprazole, brexipiprizole, and cariprazine

11. The method of claim 10, wherein at least one of the one or more genotyping assays comprises a nucleic acid amplification process followed by sequencing or gel electrophoresis of an amplification product produced thereby.

12. The method of claim 10, wherein the one or more genotyping assays determine the subject's genotype with respect to the VNTR polymorphism in the dopamine transporter DAT1/SLC6A3 gene, the rs1076560 polymorphism in the DRD2 gene, and the 48-base-pair VNTR polymorphism in the DRD4 gene.

13. The method of claim 12, wherein the one or more genotyping assays further comprise a genotyping assay that determines the subject's genome with respect to the rs4680 polymorphism in the COMT gene.

14. A method for identifying a human subject having susceptibility to a dopamine partial agonist therapy for a disorder associated with dopamine receptor activity and treating the human subject for the disorder, the method comprising:
(a) obtaining a nucleic acid sample from a human subject;
(b) performing or having performed one or more genotyping assays on a nucleic acid sample isolated from the subject to determine the subject's genotype with respect to a variable number tandem repeats (VNTR) polymorphism in a dopamine transporter DAT1/SLC6A3 gene, an rs4680 polymorphism in a DA-catabolizing enzyme catechol-O-methyltransferase (COMT) gene, an rs1076560 polymorphism in a D₂ receptor (DRD2) gene, a 48-base-pair VNTR polymorphism in a D₄ receptor (DRD4) gene, and/or an rs1800497 polymorphism in an ankyrin repeat and kinase domain containing 1 (ANKK1) gene; and
(c) administering a dopamine partial agonist to the subject if the one or more genotyping assays indicates that subject's genotype includes:
(i) at least one allele for 9 tandem repeats of the DAT1/SLC6A3 VNTR; or
(ii) four or more of: (1) a 9 tandem repeat allele of the DAT1/SLC6A3 VNTR; (2) a COMT A allele of the rs4680 polymorphism; (3) a 48-base-pair VNTR in DRD4 exon 3 allele; and (4) a DRD2 T allele of the rs1076560 polymorphism, an ANKK1 TaqA1 A allele of the rs1800497 polymorphism, or both.

15. The method of claim 14, wherein the one or more genotyping assays are performed after initiating a dopamine partial agonist therapy, and further wherein if the subject is homozygous for a VNTR 10 tandem repeat allele, the dopamine partial agonist therapy is discontinued.

16. The method of claim 14, wherein the dopamine partial agonist is selected from the group consisting of aripiprazole, brexipiprizole, and cariprazine.

17. The method of claim 14, wherein at least one of the genotyping assays comprises a nucleic acid amplification process followed by sequencing or gel electrophoresis of a resulting nucleic acid amplification product.

18. The method of claim 14, wherein the one or more genotyping assays determine the subject's genotype with respect to the VNTR polymorphism in the dopamine transporter DAT1/SLC6A3 gene, the rs1076560 polymorphism in the DRD2 gene, and the 48-base-pair VNTR polymorphism in the DRD4 gene.

19. The method of claim 18, wherein the one or more genotyping assays further comprise a genotyping assay that determines the subject's genome with respect to the rs4680 polymorphism in the COMT gene.


SEQUENCE LISTING (23 sequences)

SEQ ID NO: 1 - DNA, 23 bp, Artificial Sequence
  Artificially synthesized oligonucleotide primer
tgtggtgtagggaacggcctgag

SEQ ID NO: 2 - DNA, 24 bp, Artificial Sequence
  Artificially synthesized oligonucleotide primer
cttcctggaggtcacggctcaagg

SEQ ID NO: 3 - DNA, 18 bp, Artificial Sequence
  Artificially synthesized oligonucleotide primer
aggaccctcatggccttg

SEQ ID NO: 4 - DNA, 20 bp, Artificial Sequence
  Artificially synthesized oligonucleotide primer
gcgactacgtggtctactcg

SEQ ID NO: 5 - DNA, 40 bp, Homo sapiens
actggagcgtgtactaccccaggacgcatgcagggccccc

SEQ ID NO: 6 - DNA, 51 bp, Homo sapiens
  misc_feature (26)..(26): y = c or t
tggacgtccagctgggcgcctgcctygaccagcactttgaggatggctgtg

SEQ ID NO: 7 - DNA, 51 bp, Homo sapiens
  misc_feature (26)..(26): r = a or g
ccagcggatggtggatttcgctggcrtgaaggacaaggtgtgcatgcctga

SEQ ID NO: 8 - DNA, 51 bp, Homo sapiens
cccatctcactggcccctccctttcmccctctgaagactcctgcaaacacc

SEQ ID NO: 9 - DNA, 3952 bp, Homo sapiens
  CDS (127)..(1989)
cgctgcggagcgggaggggaggcttcgcggaacgctctcggcgccaggactcgcgtgcaaagcccaggcccgggcggccagaccaagagggaagaagcacagaattcctcaactcccagtgtgcccatgagtaagagcaaatgctccgtgggactcatgtcttccgtggtggccccggctaaggagcccaatgccgtgggcccgaaggaggtggagctcatccttgtcaaggagcagaacggagtgcagctcaccagctccaccctcaccaacccgcggcagagccccgtggaggcccaggatcgggagacctggggcaagaagatcgactttctcctgtccgtcattggctttgctgtggacctggccaacgtctggcggttcccctacctgtgctacaaaaatggtggcggtgccttcctggtcccctacctgctcttcatggtcattgctgggatgccacttttctacatggagctggccctcggccagttcaacagggaaggggccgctggtgtctggaagatctgccccatactgaaaggtgtgggcttcacggtcatcctcatctcactgtatgtcggcttcttctacaacgtcatcatcgcctgggcgctgcactatctcttctcctccttcaccacggagctcccctggatccactgcaacaactcctggaacagccccaactgctcggatgcccatcctggtgactccagtggagacagctcgggcctcaacgacacttttgggaccacacctgctgccgagtactttgaacgtggcgtgctgcacctccaccagagccatggcatcgacgacctggggcctccgcggtggcagctcacagcctgcctggtgctggtcatcgtgctgctctacttcagcctctggaagggcgtgaagacctcagggaaggtggtatggatcacagccaccatgccatacgtggtcctcactgccctgctcctgcgtggggtcaccctccctggagccatagacggcatcagagcatacctgagcgttgacttctaccggctctgcgaggcgtctgtttggattgacgcggccacccaggtgtgcttctccctgggcgtggggttcggggtgctgatcgccttctccagctacaacaagttcaccaacaactgctacagggacgcgattgtcaccacctccatcaactccctgacgagcttctcctccggcttcgtcgtcttctccttcctggggtacatggcacagaagcacagtgtgcccatcggggacgtggccaaggacgggccagggctgatcttcatcatctacccggaagccatcgccacgctccctctgtcctcagcctgggccgtggtcttcttcatcatgctgctcaccctgggtatcgacagcgccatgggtggtatggagtcagtgatcaccgggctcatcgatgagttccagctgctgcacagacaccgtgagctcttcacgctcttcatcgtcctggcgaccttcctcctgtccctgttctgcgtcaccaacggtggcatctacgtcttcacgctcctggaccattttgcagccggcacgtccatcctctttggagtgctcatcgaagccatcggagtggcctggttctatggtgttgggcagttcagcgacgacatccagcagatgaccgggcagcggcccagcctgtactggcggctgtgctggaagctggtcagcccctgctttctcctgttcgtggtcgtggtcagcattgtgaccttcagacccccccactacggagcctacatcttccccgactgggccaacgcgctgggctgggtcatcgccacatcctccatggccatggtgcccatctatgcggcctacaagttctgcagcctgcctgggtcctttcgagagaaactggcctacgccattgcacccgagaaggaccgtgagctggtggacagaggggaggtgcgccagttcacgctccgccactggctcaaggtgtagagggagcagagacgaagaccccaggaagtcatcctgcaatgggagagacacgaacaaaccaaggaaatctaagtttcgagagaaaggagggcaacttctactcttcaacctctactgaaaacacaaacaacaaagcagaagactcctctcttctgactgtttacacctttccgtgccgggagcgcacctcgccgtgtcttgtgttgctgtaataacgacgtagatctgtgcagcgaggtccaccccgttgttgtccctgcagggcagaaaaacgtctaacttcatgctgtctgtgtgaggctccctccctccctgctccctgctcccggctctgaggctgccccaggggcactgtgttctcaggcggggatcacgatccttgtagacgcacctgctgagaatccccgtgctcacagtagcttcctagaccatttactttgcccatattaaaaagccaagtgtcctgcttggtttagctgtgcagaaggtgaaatggaggaaaccacaaattcatgcaaagtcctttcccgatgcgtggctcccagcagaggccgtaaattgagcgttcagttgacacattgcacacacagtctgttcagaggcattggaggatgggggtcctggtatgtctcaccaggaaattctgtttatgttcttgcagcagagagaaataaaactccttgaaaccagctcaggctactgccactcaggcagcctgtgggtccttgcggtgtagggaacggcctgagaggagcgtgtcctatccccggacgcatgcagggcccccacaggagcgtgtcctatccccggacgcatgcagggcccccacaggagcatgtcctatccctggacgcatgcagggcccccacaggagcgtgtactaccccagaacgcatgcagggcccccacaggagcgtgtactaccccaggacgcatgcagggcccccactggagcgtgtactaccccaggacgcatgcagggcccccacaggagcgtgtcctatccccggaccggacgcatgcagggcccccacaggagcgtgtactaccccaggacgcatgcagggcccccacaggagcgtgtactaccccaggatgcatgcagggcccccacaggagcgtgtactaccccaggacgcatgcagggcccccatgcaggcagcctgcagaccacactctgcctggccttgagccgtgacctccaggaagggaccccactggaattttatttctctcaggtgcgtgccacatcaataacaacagtttttatgtttgcgaatggctttttaaaatcatatttacctgtgaatcaaaacaaattcaagaatgcagtatccgcgagcctgcttgctgatattgcagtttttgtttacaagaataattagcaatactgagtgaaggatgttggccaaaagctgctttccatggcacactgccctctgccactgacaggaaagtggatgccatagtttgaattcatgcctcaagtcggtgggcctgcctacgtgctgcccgagggcaggggccgtgcagggccagtcatggctgtcccctgcaagtggacgtgggctccagggactggagtgtaatgctcggtgggagccgtcagcctgtgaactgccaggcagctgcagttagcacagaggatggcttccccattgccttctggggagggacacagaggacggcttccccatcgccttctggccgctgcagtcagcacagagagcggcttccccattgccttctggggagggacacagaggacagcttccccatcgccttctggctgctgcagtcagcacagagagcggcttccccatcgccttctggggaggggctccgtgtagcaacccaggtgttgtccgtgtctgttgaccaatctctattcagcatcgtgtgggtccctaagcacaataaaagacatccacaatggaaaaactgcaaaaaaaaaaaaaaaaaa

SEQ ID NO: 10 - protein, 620 aa, Homo sapiens
MSKSKCSVGLMSSVVAPAKEPNAVGPKEVELILVKEQNGVQLTSSTLTNPRQSPVEAQDRETWGKKIDFLLSVIGFAVDLANVWRFPYLCYKNGGGAFLVPYLLFMVIAGMPLFYMELALGQFNREGAAGVWKICPILKGVGFTVILISLYVGFFYNVIIAWALHYLFSSFTTELPWIHCNNSWNSPNCSDAHPGDSSGDSSGLNDTFGTTPAAEYFERGVLHLHQSHGIDDLGPPRWQLTACLVLVIVLLYFSLWKGVKTSGKVVWITATMPYVVLTALLLRGVTLPGAIDGIRAYLSVDFYRLCEASVWIDAATQVCFSLGVGFGVLIAFSSYNKFTNNCYRDAIVTTSINSLTSFSSGFVVFSFLGYMAQKHSVPIGDVAKDGPGLIFIIYPEAIATLPLSSAWAVVFFIMLLTLGIDSAMGGMESVITGLIDEFQLLHRHRELFTLFIVLATFLLSLFCVTNGGIYVFTLLDHFAAGTSILFGVLIEAIGVAWFYGVGQFSDDIQQMTGQRPSLYWRLCWKLVSPCFLLFVVVVSIVTFRPPHYGAYIFPDWANALGWVIATSSMAMVPIYAAYKFCSLPGSFREKLAYAIAPEKDRELVDRGEVRQFTLRHWLKV

SEQ ID NO: 11 - DNA, 2304 bp, Homo sapiens
  CDS (250)..(1065)
cggcctgcgtccgccaccggaagcgccctcctaatccccgcagcgccaccgccattgccgccatcgtcgtggggcttctggggcagctagggctgcccgccgcgctgcctgcgccggaccggggcgggtccagtcccgggcgggccgtcgcgggagagaaataacatctgctttgctgccgagctcagaggagaccccagacccctcccgcagccagagggctggagcctgctcagaggtgctttgaagatgccggaggccccgcctctgctgttggcagctgtgttgctgggcctggtgctgctggtggtgctgctgctgcttctgaggcactggggctggggcctgtgccttatcggctggaacgagttcatcctgcagcccatccacaacctgctcatgggtgacaccaaggagcagcgcatcctgaaccacgtgctgcagcatgcggagcccgggaacgcacagagcgtgctggaggccattgacacctactgcgagcagaaggagtgggccatgaacgtgggcgacaagaaaggcaagatcgtggacgccgtgattcaggagcaccagccctccgtgctgctggagctgggggcctactgtggctactcagctgtgcgcatggcccgcctgctgtcaccaggggcgaggctcatcaccatcgagatcaaccccgactgtgccgccatcacccagcggatggtggatttcgctggcgtgaaggacaaggtcacccttgtggttggagcgtcccaggacatcatcccccagctgaagaagaagtatgatgtggacacactggacatggtcttcctcgaccactggaaggaccggtacctgccggacacgcttctcttggaggaatgtggcctgctgcggaaggggacagtgctactggctgacaacgtgatctgcccaggtgcgccagacttcctagcacacgtgcgcgggagcagctgctttgagtgcacacactaccaatcgttcctggaatacagggaggtggtggacggcctggagaaggccatctacaagggcccaggcagcgaagcagggccctgactgcccccccggcccccctctcgggctctctcacccagcctggtactgaaggtgccagacgtgctcctgctgaccttctgcggctccgggctgtgtcctaaatgcaaagcacacctcggccgaggcctgcgccctgacatgctaacctctctgaactgcaacactggattgttcttttttaagactcaatcatgacttctttactaacactggctagctatattatcttatatactaatatcatgttttaaaaatataaaatagaaattaagaatctaaatatttagatataactcgacttagtacatccttctcaactgccattcccctgctgcccttgacttgggcaccaaacattcaaagctccccttgacggacgctaacgctaagggcggggcccctagctggctgggttctgggtggcacgcctggcccactggcctcccagccacagtggtgcagaggtcagccctcctgcagctaggccaggggcacctgttagccccatggggacgactgccggcctgggaaacgaagaggagtcagccagcattcacacctttctgaccaagcaggcgctggggacaggtggaccccgcagcagcaccagcccctctgggccccatgtggcacagagtggaagcatctccttccctactccccactgggccttgcttacagaagaggcaatggctcagaccagctcccgcatccctgtagttgcctccctggcccatgagtgaggatgcagtgctggtttctgcccacctacacctagagctgtccccatctcctccaaggggtcagactgctagccacctcagaggctccaagggcccagttcccaggcccaggacaggaatcaaccctgtgctagctgagttcacctgcaccgagaccagcccctagccaagattctactcctgggctcaaggcctggctagcccccagccagcccactcctatggatagacagaccagtgagcccaagtggacaagtttggggccacccagggaccagaaacagagcctctgcaggacacagcagatgggcacctgggaccacctccacccagggccctgccccagacgcgcagaggcccgacacaagggagaagccagccacttgtgccagacctgagtggcagaaagcaaaaagttcctttgctgctttaatttttaaattttcttacaaaaatttaggtgtttaccaatagtcttattttggcttatttttaa

SEQ ID NO: 12 - protein, 271 aa, Homo sapiens
MPEAPPLLLAAVLLGLVLLVVLLLLLRHWGWGLCLIGWNEFILQPIHNLLMGDTKEQRILNHVLQHAEPGNAQSVLEAIDTYCEQKEWAMNVGDKKGKIVDAVIQEHQPSVLLELGAYCGYSAVRMARLLSPGARLITIEINPDCAAITQRMVDFAGVKDKVTLVVGASQDIIPQLKKKYDVDTLDMVFLDHWKDRYLPDTLLLEECGLLRKGTVLLADNVICPGAPDFLAHVRGSSCFECTHYQSFLEYREVVDGLEKAIYKGPGSEAGP

SEQ ID NO: 13 - DNA, 4065 bp, Homo sapiens
  CDS (1004)..(2344)
ggggtgggctgtgccccgcgggaaccccgccggcctgtgcgcttgctggtgccagctcggctcgctgcctcgcattgccacaggctcctgagaggtcgcgggcagtgcctgcggggaggcgcggggccctgctctgtagggctgaaggccgcccgaggttcgccaaggctctgggctctcgaaaggaagccaagaaaagaagctgcccaggtgaccagtcctgggagtgctctctcccaaggaagctccgagcgcccaggagcccttagccggggtctagtgccctttgaacaatctccagctcttcaaggaagtgggctgccgccgcctctcttgggacctggcctgggatcctttccccaaacgcaccccggcgatttttgcgcaccgggagccgaacccctgctgcgcgcagctggctgggctcaggcgcgcttcctcaacgtttcggagccgctgcccccagcgaagtccacattccaagctccaggggctttgagagagacgaccccaaggcaaggcgtttggagagctgctgaggagccaggggcttggaggagcgagaagacatgtattttcagctgagtctcagaaggggagaatctcctgtcaccaccagaaaagcaacagccccgaaatgtgattgcaactgactagcagagcagaggcccaggagtcactggattgatgatttagaatatgctaaaaagccagtgctttatttggggaattcaggggctttctggtgcccaagacagtgacctgcagcaagggagtcagaagacagatgtagaaatcaagagtgaccatccacgggattgacttggattgccactcaagcggtcctctcatggaatgttggtgaggccctctgccagggaagcaatctggctgtgcaaagtgctgcctggtggggaggactcctggaaatctgactgacccctattccctgcttgggaacttgaggggtgtcagagcccctgatgtgctttctcttaggaagatgaggactctgaacacctctgccatggacgggactgggctggtggtggagagggacttctctgttcgtatcctcactgcctgtttcctgtcgctgctcatcctgtccacgctcctggggaacacgctggtctgtgctgccgttatcaggttccgacacctgcggtccaaggtgaccaacttctttgtcatctccttggctgtgtcagatctcttggtggccgtcctggtcatgccctggaaggcagtggctgagattgctggcttctggccctttgggtccttctgtaacatctgggtggcctttgacatcatgtgctccactgcatccatcctcaacctctgtgtgatcagcgtggacaggtattgggctatctccagccctttccggtatgagagaaagatgacccccaaggcagccttcatcctgatcagtgtggcatggaccttgtctgtactcatctccttcatcccagtgcagctcagctggcacaaggcaaaacccacaagcccctctgatggaaatgccacttccctggctgagaccatagacaactgtgactccagcctcagcaggacatatgccatctcatcctctgtaataagcttttacatccctgtggccatcatgattgtcacctacaccaggatctacaggattgctcagaaacaaatacggcgcattgcggccttggagagggcagcagtccacgccaagaattgccagaccaccacaggtaatggaaagcctgtcgaatgttctcaaccggaaagttcttttaagatgtccttcaaaagagaaactaaagtcctgaagactctgtcggtgatcatgggtgtgtttgtgtgctgttggctacctttcttcatcttgaactgcattttgcccttctgtgggtctggggagacgcagcccttctgcattgattccaacacctttgacgtgtttgtgtggtttgggtgggctaattcatccttgaaccccatcatttatgcctttaatgctgattttcggaaggcattttcaaccctcttaggatgctacagactttgccctgcgacgaataatgccatagagacggtgagtatcaataacaatggggccgcgatgttttccagccatcatgagccacgaggctccatctccaaggagtgcaatctggtttacctgatcccacatgctgtgggctcctctgaggacctgaaaaaggaggaggcagctggcatcgccagacccttggagaagctgtccccagccctatcagtcatattggactatgacactgacgtctctctggagaagatccaacccatcacacaaaacggtcagcacccaacctgaactcgcagatgaatcctgccacacatgctcatcccaaaagctagaggagattgctctggggcttgctattaagaaactaaggtacggtgagactctgaggtgtcaggagagccctctgctgctttccaacacacaattaactccgtttccaaatacattccagtgtattttctgtgttgttcatagtcaatcaaacagggacactacaaacatggggagccataagggacatgtctttggcttcagaattgtttttagaaatttattcttatcttaggatttaccaaatagggcaaagaatcaacagtgaacagcttcacttaaaatcaaatttttctgggaagaaaatgagatgggttgagtttgctgtatacaaacaggtgctaacactgttcccagcaaagttttcagattgtaaaggtaggtgcatgccttcataaattatttctaaaacattaattgaggcttacagtaggagtgagaaatttttttccagaattgagagatgttttgttgatattggttctatttatttattgtatatatggatatttttaatttatgatataataaatatatatttatcatatttaataggataaattaatgagttttatccaagaccttacaaccacatttctggccatttaactagcactttataagccaatgaagcaaacacacagactctgtgagattctaaatgttcatgtgtaacttctagaaacacagcagaaactgatagataagggaataaagttgaaatgattccttaaaattcatggacacagataaatgcaaggtgagaattgacaaatgctataaatgctttctttttctgaaaagattttgaaaaatttaaaaaagtatagctactactgtgttcaaaacgttttaaatgacaaatgactttcccaggggaatttgcagttctgtaaatatcttaaataaaagccaacttaagaagagcccagcattaaatttacgatcttaggtggtaatgaaaagtatatgctgctttgtatttatgtaaaataattggccctctccatcttttctcatttcatgtgtcaggtagtttttctgaaccacacaaatggctttcctggagagagatctgtagcacagacagtgggttacagcagccccactgagggaccaaactcaaaccctgcatttccatcttaccaggtcaaaccaaaccagtcagtggggctactttttatagtgctttaatctgaatttagagctgatttttaaaggagtctttaaatgttaatggtatactaactaacgaatagtgcctcattatcattcttgagtcagatacttctgttgatgggagaaacagaagaatccttccctttgggtgtgttgagctcccccaaagccatcagcatctcttttgacaaatgctagtcctttctctgtgctttggaatcaggttcctgcatcatcacccggactgtaaaaagtatcataagcctcccttgccagatgccaactcgtggggcatttcaacagagtttctttgaaatgtttacaacgtattcttcttgataagcaatgaacttaacatttagatgcaatccgtgaaaagaaaaaaaaatctgaaaaatatctcctgcatcaggtctgtgttatttatgtattgtgaatgttttcttaattttattggctgtatgctttcttacacataataaaaatattttgtgaactca

SEQ ID NO: 14 - protein, 446 aa, Homo sapiens
MRTLNTSAMDGTGLVVERDFSVRILTACFLSLLILSTLLGNTLVCAAVIRFRHLRSKVTNFFVISLAVSDLLVAVLVMPWKAVAEIAGFWPFGSFCNIWVAFDIMCSTASILNLCVISVDRYWAISSPFRYERKMTPKAAFILISVAWTLSVLISFIPVQLSWHKAKPTSPSDGNATSLAETIDNCDSSLSRTYAISSSVISFYIPVAIMIVTYTRIYRIAQKQIRRIAALERAAVHAKNCQTTTGNGKPVECSQPESSFKMSFKRETKVLKTLSVIMGVFVCCWLPFFILNCILPFCGSGETQPFCIDSNTFDVFVWFGWANSSLNPIIYAFNADFRKAFSTLLGCYRLCPATNNAIETVSINNNGAAMFSSHHEPRGSISKECNLVYLIPHAVGSSEDLKKEEAAGIARPLEKLSPALSVILDYDTDVSLEKIQPITQNGQHPT

SEQ ID NO: 15 - DNA, 2713 bp, Homo sapiens
  CDS (236)..(1567)
actgctccccgcgggccagagccggccgagctgctgcccgccggggctctgaacggcgcggcggggccgggagccagggaccggccgaggagagtggcggccccggacggctgccggaggggcggccgcgcgtggatgcggcgggagctggaagcctcaagcagccggcgccgtctctgcccccgggcgccctatggcttgaagagcctggccacccagtggctccaccgccctgatggatccactgaatctgtcctggtatgatgatgatctggagaggcagaactggagccggcccttcaacgggtcagacgggaaggcggacagaccccactacaactactatgccacactgctcaccctgctcatcgctgtcatcgtcttcggcaacgtgctggtgtgcatggctgtgtcccgcgagaaggcgctgcagaccaccaccaactacctgatcgtcagcctcgcagtggccgacctcctcgtcgccacactggtcatgccctgggttgtctacctggaggtggtaggtgagtggaaattcagcaggattcactgtgacatcttcgtcactctggacgtcatgatgtgcacggcgagcatcctgaacttgtgtgccatcagcatcgacaggtacacagctgtggccatgcccatgctgtacaatacgcgctacagctccaagcgccgggtcaccgtcatgatctccatcgtctgggtcctgtccttcaccatctcctgcccactcctcttcggactcaataacgcagaccagaacgagtgcatcattgccaacccggccttcgtggtctactcctccatcgtctccttctacgtgcccttcattgtcaccctgctggtctacatcaagatctacattgtcctccgcagacgccgcaagcgagtcaacaccaaacgcagcagccgagctttcagggcccacctgagggctccactaaagggcaactgtactcaccccgaggacatgaaactctgcaccgttatcatgaagtctaatgggagtttcccagtgaacaggcggagagtggaggctgcccggcgagcccaggagctggagatggagatgctctccagcaccagcccacccgagaggacccggtacagccccatcccacccagccaccaccagctgactctccccgacccgtcccaccatggtctccacagcactcccgacagccccgccaaaccagagaagaatgggcatgccaaagaccaccccaagattgccaagatctttgagatccagaccatgcccaatggcaaaacccggacctccctcaagaccatgagccgtaggaagctctcccagcagaaggagaagaaagccactcagatgctcgccattgttctcggcgtgttcatcatctgctggctgcccttcttcatcacacacatcctgaacatacactgtgactgcaacatcccgcctgtcctgtacagcgccttcacgtggctgggctatgtcaacagcgccgtgaaccccatcatctacaccaccttcaacattgagttccgcaaggccttcctgaagatcctccactgctgactctgctgcctgcccgcacagcagcctgcttcccacctccctgcccaggccggccagcctcacccttgcgaaccgtgagcaggaaggcctgggtggatcggcctcctcttcaccccggcaggccctgcagtgttcgcttggctccatgctcctcactgcccgcacaccctcactctgccagggcagtgctagtgagctgggcatggtaccagccctggggctgggccccccagctcaggggcagctcatagagtcccccctcccacctccagtccccctatccttggcaccaaagatgcagccgccttccttgaccttcctctggggctctagggttgctggagcctgagtcagggcccagaggctgagttttctctttgtggggcttggcgtggagcaggcggtggggagagatggacagttcacaccctgcaaggcccacaggaggcaagcaagctctcttgccgaggagccaggcaacttcagtcctgggagacccatgtaaataccagactgcaggttggaccccagagattcccaagccaaaaaccttagctccctcccgcaccccgatgtggacctctactttccaggctagtccggacccacctcaccccgttacagctccccaagtggtttccacatgctctgagaagaggagccctcatcttgaagggcccaggagggtctatggggagaggaactccttggcctagcccaccctgctgccttctgacggccctgcaatgtatcccttctcacagcacatgctggccagcctggggcctggcagggaggtcaggccctggaactctatctgggcctgggctaggggacatcagaggttctttgagggactgcctctgccacactctgacgcaaaaccactttccttttctattccttctggcctttcctctctcctgtttcccttcccttccactgcctctgccttagaggagcccacggctaagaggctgctgaaaaccatctggcctggcctggccctgccctgaggaaggaggggaagctgcagcttgggagagcccctggggcctagactctgtaacatcactatccatgcaccaaactaataaaactttgacgagtcaccttccaggacccctgggtaaaaaaaaaaaaaaa

SEQ ID NO: 16 - protein, 443 aa, Homo sapiens
MDPLNLSWYDDDLERQNWSRPFNGSDGKADRPHYNYYATLLTLLIAVIVFGNVLVCMAVSREKALQTTTNYLIVSLAVADLLVATLVMPWVVYLEVVGEWKFSRIHCDIFVTLDVMMCTASILNLCAISIDRYTAVAMPMLYNTRYSSKRRVTVMISIVWVLSFTISCPLLFGLNNADQNECIIANPAFVVYSSIVSFYVPFIVTLLVYIKIYIVLRRRRKRVNTKRSSRAFRAHLRAPLKGNCTHPEDMKLCTVIMKSNGSFPVNRRRVEAARRAQELEMEMLSSTSPPERTRYSPIPPSHHQLTLPDPSHHGLHSTPDSPAKPEKNGHAKDHPKIAKIFEIQTMPNGKTRTSLKTMSRRKLSQQKEKKATQMLAIVLGVFIICWLPFFITHILNIHCDCNIPPVLYSAFTWLGYVNSAVNPIIYTTFNIEFRKAFLKILHC

SEQ ID NO: 17 - DNA, 1698 bp, Homo sapiens
  CDS (432)..(1634)
gatagagagacaaagaggaaaagagagcgaggtagaaaacggatactgcctatgcctactccatccctctttcagcaccaaggacagaacctctgagcggctgacccaagcaacgctcagtttagggtccctcccaaatcctctaaagaaaacggatacattcgaaagcagctatgaaacatgcactaaggtctaatagggaagctggaaaagcagcactcaagtaatttcaccttagaggcaaaaatgggtgatttctttctgttcatttcatagtttctgagtcctgagaaaggcaaagtttgctttgcttgggtatgtctgctgtcagtaaatggctgcaggagccgaagtggtaaactcctcggtctccagaaatcagaagaaaattttagggaagccccttggcatcacgcacctccctctgggctatggcatctctgagccagctgagtggccacctgaactacacctgtggggcagagaactccacaggtgccagccaggcccgcccacatgcctactatgccctctcctactgcgcgctcatcctggccatcgtcttcggcaatggcctggtgtgcatggctgtgctgaaggagcgggccctgcagactaccaccaactacttagtagtgagcctggctgtggcagacttgctggtggccaccttggtgatgccctgggtggtatacctggaggtgacaggtggagtctggaatttcagccgcatttgctgtgatgtttttgtcaccctggatgtcatgatgtgtacagccagcatccttaatctctgtgccatcagcatagacaggtacactgcagtggtcatgcccgttcactaccagcatggcacgggacagagctcctgtcggcgcgtggccctcatgatcacggccgtctgggtactggcctttgctgtgtcctgccctcttctgtttggctttaataccacaggggaccccactgtctgctccatctccaaccctgattttgtcatctactcttcagtggtgtccttctacctgccctttggagtgactgtccttgtctatgccagaatctatgtggtgctgaaacaaaggagacggaaaaggatcctcactcgacagaacagtcagtgcaacagtgtcaggcctggcttcccccaacaaaccctctctcctgacccggcacatctggagctgaagcgttactacagcatctgccaggacactgccttgggtggaccaggcttccaagaaagaggaggagagttgaaaagagaggagaagactcggaattccctgagtcccaccatagcgcccaagctcagcttagaagttcgaaaactcagcaatggcagattatcgacatctttgaagctggggcccctgcaacctcggggagtgccacttcgggagaagaaggcaacccaaatggtggccattgtgcttggggccttcattgtctgctggctgcccttcttcttgacccatgttctcaatacccactgccagacatgccacgtgtccccagagctttacagtgccacgacatggctgggctacgtgaatagcgccctcaaccctgtgatctataccaccttcaatatcgagttccggaaagccttcctcaagatcctgtcttgctgagggagcagaagagggaacactctttgtacccatttctagctgccaggctgttggcccactcaga

SEQ ID NO: 18 - protein, 400 aa, Homo sapiens
MASLSQLSGHLNYTCGAENSTGASQARPHAYYALSYCALILAIVFGNGLVCMAVLKERALQTTTNYLVVSLAVADLLVATLVMPWVVYLEVTGGVWNFSRICCDVFVTLDVMMCTASILNLCAISIDRYTAVVMPVHYQHGTGQSSCRRVALMITAVWVLAFAVSCPLLFGFNTTGDPTVCSISNPDFVIYSSVVSFYLPFGVTVLVYARIYVVLKQRRRKRILTRQNSQCNSVRPGFPQQTLSPDPAHLELKRYYSICQDTALGGPGFQERGGELKREEKTRNSLSPTIAPKLSLEVRKLSNGRLSTSLKLGPLQPRGVPLREKKATQMVAIVLGAFIVCWLPFFLTHVLNTHCQTCHVSPELYSATTWLGYVNSALNPVIYTTFNIEFRKAFLKILSC

SEQ ID NO: 19 - DNA, 1378 bp, Homo sapiens
  CDS (1)..(1260)
atggggaaccgcagcaccgcggacgcggacgggctgctggctgggcgcgggccggccgcgggggcatctgcgggggcatctgcggggctggctgggcagggcgcggcggcgctggtggggggcgtgctgctcatcggcgcggtgctcgcggggaactcgctcgtgtgcgtgagcgtggccaccgagcgcgccctgcagacgcccaccaactccttcatcgtgagcctggcggccgccgacctcctcctcgctctcctggtgctgccgctcttcgtctactccgaggtccagggtggcgcgtggctgctgagcccccgcctgtgcgacgccctcatggccatggacgtcatgctgtgcaccgcctccatcttcaacctgtgcgccatcagcgtggacaggttcgtggccgtggccgtgccgctgcgctacaaccggcagggtgggagccgccggcagctgctgctcatcggcgccacgtggctgctgtccgcggcggtggcggcgcccgtactgtgcggcctcaacgacgtgcgcggccgcgaccccgccgtgtgccgcctggaggaccgcgactacgtggtctactcgtccgtgtgctccttcttcctaccctgcccgctcatgctgctgctctactgggccacgttccgcggcctgcagcgctgggaggtggcacgtcgcgccaagctgcacggccgcgcgccccgccgacccagcggccctggcccgccttcccccacgccacccgcgccccgcctcccccaggacccctgcggccccgactgtgcgccccccgcgcccggccttccccggggtccctgcggccccgactgtgcgcccgccgcgcccagcctcccccaggacccctgcggccccgactgtgcgccccccgcgcccggcctccccccggacccctgcggctccaactgtgctccccccgacgccgtcagagccgccgcgctcccaccccagactccaccgcagacccgcaggaggcggcgtgccaagatcaccggccgggagcgcaaggccatgagggtcctgccggtggtggtcggggccttcctgctgtgctggacgcccttcttcgtggtgcacatcacgcaggcgctgtgtcctgcctgctccgtgcccccgcggctggtcagcgccgtcacctggctgggctacgtcaacagcgccctcaaccccgtcatctacactgtcttcaacgccgagttccgcaacgtcttccgcaaggccctgcgtgcctgctgctgagccgggcacccccggacgccccccggcctgatggccaggcctcagggaccaaggagatggggagggcgcttttgtacgttaattaaacaaattccttcccaaaaaaaaaaaaaaaaaa

SEQ ID NO: 20 - protein, 419 aa, Homo sapiens
MGNRSTADADGLLAGRGPAAGASAGASAGLAGQGAAALVGGVLLIGAVLAGNSLVCVSVATERALQTPTNSFIVSLAAADLLLALLVLPLFVYSEVQGGAWLLSPRLCDALMAMDVMLCTASIFNLCAISVDRFVAVAVPLRYNRQGGSRRQLLLIGATWLLSAAVAAPVLCGLNDVRGRDPAVCRLEDRDYVVYSSVCSFFLPCPLMLLLYWATFRGLQRWEVARRAKLHGRAPRRPSGPGPPSPTPPAPRLPQDPCGPDCAPPAPGLPRGPCGPDCAPAAPSLPQDPCGPDCAPPAPGLPPDPCGSNCAPPDAVRAAALPPQTPPQTRRRRRAKITGRERKAMRVLPVVVGAFLLCWTPFFVVHITQALCPACSVPPRLVSAVTWLGYVNSALNPVIYTVFNAEFRNVFRKALRACC

SEQ ID NO: 21 - DNA, 2543 bp, Homo sapiens
  CDS (95)..(2392)
cggacccgaggagcaggaagcggcggctccttcggccacccaggcagcagccacagcggggagtgcgcggcgcggggacaggaagagaggggcaatggctgccgaccccaccgagctgcggctgggcagcctccccgtcttcacccgcgacgacttcgagggcgactggcgcctagtggccagcggcggcttcagccaggtgttccaggcgcggcacaggcgctggcggacggagtacgccatcaagtgcgccccctgccttccacccgacgccgccagctctgatgtgaattacctcattgaagaagctgccaaaatgaagaagatcaagtttcagcacatcgtgtctatctacggggtgtgcaagcagcccctgggtattgtgatggagtttatggccaacggctccctggagaaggtgctgtccacccacagcctctgctggaagctcaggttccgcatcatccatgagaccagcttggccatgaacttcctgcacagcattaagccgcctctgctccacctggacctcaagccgggcaacatactcctggacagcaacatgcatgtcaaaatttcagacttcggcctgtccaagtggatggaacagtccacccggatgcagtacatcgagaggtcggctctgcggggcatgctcagctacatcccccctgagatgttcctggagagtaacaaggccccaggacctaaatatgatgtgtacagctttgcaattgtcatctgggagctactcactcagaagaaaccatactcagggttcaacatgatgatgattattatccgagtggcggcaggcatgcggccctccctacagcctgtctctgaccaatggccaagcgaggcccagcagatggtggacctgatgaaacgctgctgggaccaggaccccaagaagaggccatgctttctagacattaccatcgagacagacatactgctgtcactgctgcagagtcgtgtggcagtcccagagagcaaggccctggccaggaaggtgtcctgcaagctgtcgctgcgccagcccggggaggttaatgaggacatcagccaggaactgatggacagtgactcaggaaactacctgaagcgggcccttcagctctccgaccgtaagaatttggtcccgagagatgaggaactgtgtatctatgagaacaaggtcacccccctccacttcctggtggcccagggcagtgtggagcaggtgaggttgctgctggcccacgaggtagacgtggactgccagacggcctctggatacacgcccctcctgatcgccgcccaggaccagcaacccgacctctgtgccctgcttttggcacatggtgctgatgccaaccgagtggatgaggatggctgggccccactgcactttgcagcccagaatggggatgacggcactgcgcgcctgctcctggaccacggggcctgtgtggatgcccaggaacgtgaagggtggacccctcttcacctggctgcacagaataactttgagaatgtggcacggcttctggtctcccgtcaggctgaccccaacctgcatgaggctgagggcaagacccccctccatgtggccgcctactttggccatgttagcctggtcaagctgctgaccagccagggggctgagttggatgctcagcagagaaacctgagaacaccactgcacctggcagtagagcggggcaaagtgagggccatccaacacctgctgaagagtggagcggtccctgatgcccttgaccagagcggctacggcccactgcacactgcagctgccaggggcaaatacctgatctgcaagatgctgctcaggtacggagccagccttgagctgcccacccaccagggctggacacccctgcatctagcagcctacaagggccacctggagatcatccatctgctggcagagagccacgcaaacatgggtgctcttggagctgtgaactggactcccctgcacctagctgcacgccacggggaggaggcggtggtgtcagcactgctgcagtgtggggctgaccccaatgctgcagagcagtcaggctggacacccctccacctggcggtccagaggagcaccttcctgagtgtcatcaacctcctagaacatcacgcaaatgtccacgcccgcaacaaggtgggctggacacccgcccacctggccgccctcaagggcaacacagccatcctcaaagtgctggtcgaggcaggcgcccagctggacgtccaggatggagtgagctgcacacccctgcaactggccctccgcagccgaaagcagggcatcatgtccttcctagagggcaaggagccgtcagtggccactctgggtggttctaagccaggagccgagatggaaatttagacaacttggccagccgtggtggctcacgtctgtaatcccagcactttgggaggctgaggcaggcagatcacctgatatcaagagtttgaggccagcctggccaacatggcaaaaccctgtctctgctaaaaatacaaaatttagctgggta

SEQ ID NO: 22 - protein, 765 aa, Homo sapiens
MAADPTELRLGSLPVFTRDDFEGDWRLVASGGFSQVFQARHRRWRTEYAIKCAPCLPPDAASSDVNYLIEEAAKMKKIKFQHIVSIYGVCKQPLGIVMEFMANGSLEKVLSTHSLCWKLRFRIIHETSLAMNFLHSIKPPLLHLDLKPGNILLDSNMHVKISDFGLSKWMEQSTRMQYIERSALRGMLSYIPPEMFLESNKAPGPKYDVYSFAIVIWELLTQKKPYSGFNMMMIIIRVAAGMRPSLQPVSDQWPSEAQQMVDLMKRCWDQDPKKRPCFLDITIETDILLSLLQSRVAVPESKALARKVSCKLSLRQPGEVNEDISQELMDSDSGNYLKRALQLSDRKNLVPRDEELCIYENKVTPLHFLVAQGSVEQVRLLLAHEVDVDCQTASGYTPLLIAAQDQQPDLCALLLAHGADANRVDEDGWAPLHFAAQNGDDGTARLLLDHGACVDAQEREGWTPLHLAAQNNFENVARLLVSRQADPNLHEAEGKTPLHVAAYFGHVSLVKLLTSQGAELDAQQRNLRTPLHLAVERGKVRAIQHLLKSGAVPDALDQSGYGPLHTAAARGKYLICKMLLRYGASLELPTHQGWTPLHLAAYKGHLEIIHLLAESHANMGALGAVNWTPLHLAARHGEEAVVSALLQCGADPNAAEQSGWTPLHLAVQRSTFLSVINLLEHHANVHARNKVGWTPAHLAALKGNTAILKVLVEAGAQLDVQDGVSCTPLQLALRSRKQGIMSFLEGKEPSVATLGGSKPGAEMEI

SEQ ID NO: 23 - DNA, 23 bp, Artificial Sequence
  Artificially synthesized oligonucleotide primer
tgcggtgtagggaacggcctgag